(19) United States
(12) Patent Application Publication
(10) Pub. No.: US 2026/0245450 A1
(43) Pub. Date: Aug. 20, 2026

(54) CRANIAL HUB, SYSTEMS AND METHODS OF USING SAME

(71) Applicant: Paradromics, Inc., Austin, TX (US)

(72) Inventors: Matthew R ANGLE, Austin, TX (US); James William BURROWS, Austin, TX (US); Michael LANDRY, Austin, TX (US); Kurtis Alan NISHIMURA, Austin, TX (US); Aleksander TADIC, Austin, TX (US); Kunal SAHASRABUDDHE, Austin, TX (US); Yifan KONG, Austin, TX (US); Aamir Ahmed KHAN, Austin, TX (US); Ken CHIANG, Austin, TX (US); Jacob STEGMAN, Austin, TX (US)

(73) Assignee: Paradromics, Inc., Austin, TX (US)

(21) Appl. No.: 19/179,904

(22) Filed: Apr. 15, 2025

Related U.S. Application Data
(63) Continuation of application No. PCT/US2023/077021, filed on Oct. 16, 2023.
(60) Provisional application No. 63/379,824, filed on Oct. 17, 2022.

(51) Int. Cl. G08C 17/00; A61B 5/00

(52) U.S. Cl. CPC G08C 17/00; A61B 5/0002; A61B 5/0031

(57) ABSTRACT

Described is an implantable cranial hub device having a design allowing it to reside within the profile of a human skull with minimal to no protrusion above the outer surface of the skull. The device may include a hermetically sealed package for electronics such that the device is biocompatible with a human body. Such a device provides an ability to communicate data associated with signals from a human brain.

### CROSS-REFERENCE TO RELATED APPLICATIONS

The present application is a continuation of PCT/US2023/077021 filed Oct. 19, 2023 which claims priority to U.S. Provisional Application No. 63/379,824, filed Oct. 17, 2022, which is incorporated herein by reference in its entirety.

### BACKGROUND

Many unmet medical challenges require the need for systems that can record, process, and transmit large amounts of neural signals associated with the human brain, in parallel. Additionally, systems are needed for stimulation of human brains. Such systems may be utilized for neuroscience applications and implantable brain computer interfaces (BCI).

### SUMMARY

Described herein are various embodiments of an implantable cranial hub device comprising: a housing comprising a curve, the curve comprising a radius of curvature; and a plurality of connector rows, wherein the plurality of connector rows is configured to operably couple to a plurality of implantable cortical modules and at least one wireless transmitter module; and electronic circuitry configured to (1) aggregate a plurality of signals received from the plurality of implantable cortical modules and (2) transmit the aggregated plurality of signals to the at least one wireless transmitter. In some embodiments, the radius of curvature is about 80 mm to about 120 mm. In some embodiments, the radius of curvature is about 100 mm. In some embodiments, the curve comprises at least one portion that is bent and at least one portion that is flat. In some embodiments, the at least one portion that is bent comprises a bend angle, θ. In some embodiments, the bend angle, θ, is about 15° to 20°. In some embodiments, the bend angle, θ, is about 18°. In some embodiments, the device further comprises a frame. In some embodiments, the frame is configured to permit the cranial hub to be mounted to a skull of a subject.

Described herein are various embodiments of an implantable system comprising an implantable cranial hub device and at least one wireless transmitter.

In some embodiments, the cranial hub device further comprises a plurality of the cortical modules. In some embodiments, the cranial hub device further comprises a sensor, the sensor configured to receive signals from a passive cortical module.

Described herein are various embodiments of a direct data interface (DDI) system, the system comprising: at least one cortical module; at least one implantable cranial hub device; and an implantable wireless transmitter.

Described herein are various embodiments of an implantable cranial hub device comprising: a housing comprising a layer, wherein the layer is configured to minimize heat transfer to the interior of a skull of a subject and/or maximize heat loss to the exterior of the skull of a subject; and a plurality of connector rows, wherein the plurality of connector rows is configured to operably couple to a plurality of implantable cortical modules and at least one wireless transmitter module; and electronic circuitry configured to (1) aggregate a plurality of signals received from the plurality of implantable cortical modules and (2) transmit the aggregated plurality of signals to the at least one wireless transmitter. In some embodiments, the layer is in contact with the interior of the skull of the subject and the layer is configured to block heat transfer to the interior of the skull of the subject. In some embodiments, the layer is a heat blocking layer. In some embodiments, the heat blocking layer comprises a heat blocking material. In some embodiments, the heat blocking material comprises a metal, a ceramic or a polymer. In some embodiments, the heat blocking material comprises titanium. In some embodiments, the heat blocking material comprises polyethylene polymer or PEEK polymer. In some embodiments, the heat blocking material comprises an alumina ceramic or a zirconia ceramic. In some embodiments, the heat blocking material comprises silicon carbide. In some embodiments, the layer is exposed to the exterior of the skull of the subject and the layer is configured to transfer heat to the exterior of the skull of the subject. In some embodiments, the layer is a heat transfer layer. In such embodiments, the heat transfer layer comprises a heat transfer material. In some embodiments, the heat transfer material is a metal. In some embodiments, the heat transfer material is titanium metal or stainless steel metal.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a direct data interface (DDI) system, in accordance with some embodiments;

FIG. 2 depicts a schematic diagram of the DDI system of FIG. 1, in accordance with some embodiments;

FIGS. 3A-3B depict a three-dimensional view and a side view of a cranial hub, respectively, in accordance with some embodiments;

FIG. 4 depicts an exploded-view diagram of the cranial hub, in accordance with some embodiments;

FIGS. 5A-5B depict two embodiments of the cranial hub, which can include flanges for attaching the cranial hub to a skull of a subject, in accordance with some embodiments;

FIG. 6 shows a schematic of an adult human skull along with a superimposed side profile diagram of the cranial hub, in accordance with some embodiments;

FIGS. 7A-7C depict scaled drawings of a cranial hub, in accordance with some embodiments;

FIGS. 8A-8B depict schematics for a set screw assembly of the connector row in, accordance with some embodiments;

FIG. 9 depicts a cross sectional diagram of a cranial hub including a heat transfer layer and heat blocking layer in accordance with some embodiments;

FIGS. 10A-10C depict various profiles of the cranial hub housing, in accordance with some embodiments;

FIGS. 11A-11B depict various embodiments of an implanted cortical module, in accordance with some embodiments;

FIG. 12 depicts a diagram of a parallel array of discrete event detectors (e.g., sensor array) of a cortical module, in accordance with some embodiments;

FIG. 13 depicts a cranial hub design with a wireless transmitter (e.g., transceiver) interface, in accordance with some embodiments;

FIG. 14 illustrates the curve and radius of curvature for a cranial hub, in accordance with some embodiments;

FIG. 15 depicts multiple passive cortical modules linked to a cranial hub, in accordance with some embodiments;

FIG. 16 depicts a cranial hub having a hermetic feedthrough configured to connect to individual wires (e.g., electrodes) of one or more passive cortical modules, in accordance with some embodiments;

FIG. 17 depicts a cranial hub including a frame with flanges that can be configured to attach the cranial hub to a skull of a subject, in accordance with some embodiments;

FIGS. 18A-18C depict a cranial hub including another frame with flanges that can be configured to attach the cranial hub to a skull of a subject, in accordance with some embodiments;

FIGS. 19A-19D depict a cranial hub including a flexible frame with flanges that can include formable flanges for attaching the cranial hub to a skull of a subject, in accordance with some embodiments;

FIGS. 20A-20C depict a cranial hub including another frame with flanges that can be configured to attach the cranial hub to a skull of a subject, in accordance with some embodiments;

FIGS. 21A-21E depict a cranial hub including another frame with flanges that can be configured to attach the cranial hub to a skull of a subject, in accordance with some embodiments;

FIGS. 22A-22B depict cranial hub including another frame with flanges that can be configured to attach the cranial hub to a skull of a subject, in accordance with some embodiments;

FIGS. 23A-23B depict a cranial hub including another frame with flanges that can be configured to attach the cranial hub to a skull of a subject, in accordance with some embodiments;

FIGS. 24A-24B depict a cranial hub including another frame with flanges that can be configured to attach the cranial hub to a skull of a subject, in accordance with some embodiments;

FIGS. 25A-25B depict a cranial hub including another frame with and flanges that can be configured to attach the cranial hub to a skull of a subject, in accordance with some embodiments;

FIG. 26 depicts a cranial hub including another frame flanges that can be configured to attach the cranial hub to a skull of a subject, in accordance with some embodiments;

FIG. 27 depicts a cranial hub including another frame with flanges that can be configured to attach the cranial hub to a skull of a subject, in accordance with some embodiments; and

FIGS. 28A-28B depict a cranial hub including another frame with flanges that can be configured to attach the cranial hub to a skull of a subject, in accordance with some embodiments.

### DETAILED DESCRIPTION

### Introduction

While various embodiments have been shown and described herein, it will be obvious to those skilled in the art that such embodiments are provided by way of example only. Numerous variations, changes, and substitutions may occur to those skilled in the art without departing from the present disclosure. It should be understood that various alternatives to the embodiments described herein may be employed.

The present disclosure relates to a cranial hub configured for aggregating (e.g., coalescing) detected signals of bioelectric activity from hundreds, thousands, tens of thousands, hundreds of thousands, or millions of neurons and efficiently encoding the data stream associated with that activity in real-time. In some embodiments, a cortical module utilizes a massively parallel array of electronic detectors (e.g., a sensor array) to record important features of neural activity (e.g., signals of bioelectric activity). In some embodiments, a cranial hub aggregates (e.g., coalesces) these detected features into discrete bioelectric events or epochs based on a prior model of both the neural signal source and feature extraction circuitry. Finally, in some cases, a cranial hub may find alternative representations (or encodings) of this neural activity data that accomplish compression and/or dimensional reduction. These operations may be accomplished in a manner that reduces bandwidth and energy requirements, promotes miniaturization of the technology, and avoids excessive heating of neural tissue. In some embodiments, the cranial hub, as part of the DDI system, may assist in stimulating neural activity.

The human brain is composed of nearly 100 billion neurons. Each neuron generates signals in the form of time-varying electrochemical potentials across their cellular membrane (action potentials). Due to its electrochemical nature, the timescale on which a single neural event takes place is orders of magnitude slower than electronic interactions implemented in a computer. Nonetheless, the human brain can leverage the interactions of many neurons to perform highly parallel computations. These complex signal cascades are responsible for all the brain's information processing capabilities.

While neural circuits require the parallel action of many thousands to millions of individual neurons, currently known methods for studying such circuits are typically only capable of measuring individual neural signals from a limited number of cells at a time. This limits knowledge of how networks of many neurons give rise to the wide range of tasks performed by the brain. Thus, there is a need for systems, devices, and methods for detecting and processing neural events from a very large number of individual neurons. The results of the neural processing may provide a more in-depth understanding of how the brain functions. Such an understanding may allow treatment of disorders of the nervous system such as blindness, paralysis, and neurodegenerative diseases. Additionally, increasing the scale of interaction between man-made electronics and the human brain could lead to a new generation of high data rate brain-machine interfaces (BMIs) that may control complex prosthetics or mediate sensory input to the brain from devices such as digital cameras or microphones.

Processing information from a large number of individual neurons is a technological challenge due to the large amount of information generated. Continuous analog-to-digital conversion (ADC) of each neural action potential typically requires ADCs with high bit depths and sample rates. To process information from many hundreds, thousands, tens of thousands, hundreds of thousands, or millions of neurons, bulky electronics and cables may be required, which may restrict body movement. In other instances, attempts to transmit fully sampled signals wirelessly from an implanted neural probe using current wireless technologies may cause undesirable heating of electronic circuits in the neural probe, and possibly result in damage to neural tissue. Therefore, there is a need for direct data interface (DDI) systems devices, and methods for detecting and processing neural events from a very large number of neurons, in a manner that minimizes the amount of information transmitted and heating emitted from an implanted device while retaining enough information to generate useful data about the signals generated by many thousands to millions of neurons. In some embodiments, devices configured to receive neural events (e.g., neural signals) from a very large number of neurons, process those signals and transmit processed signals to a wireless transmitter may be referred to as cortical modules, cranial hubs or a combination thereof. In some embodiments, cranial hubs share characteristics in whole, or in part, with feature extraction modules and/or a feature-event coalescence modules as described in U.S. Patent Application Publication No. 2019/0246929, incorporated herein by reference in its entirety. Further, there is a need for minimizing the amount of protrusion of cranial hub devices implanted in the skull of a subject.

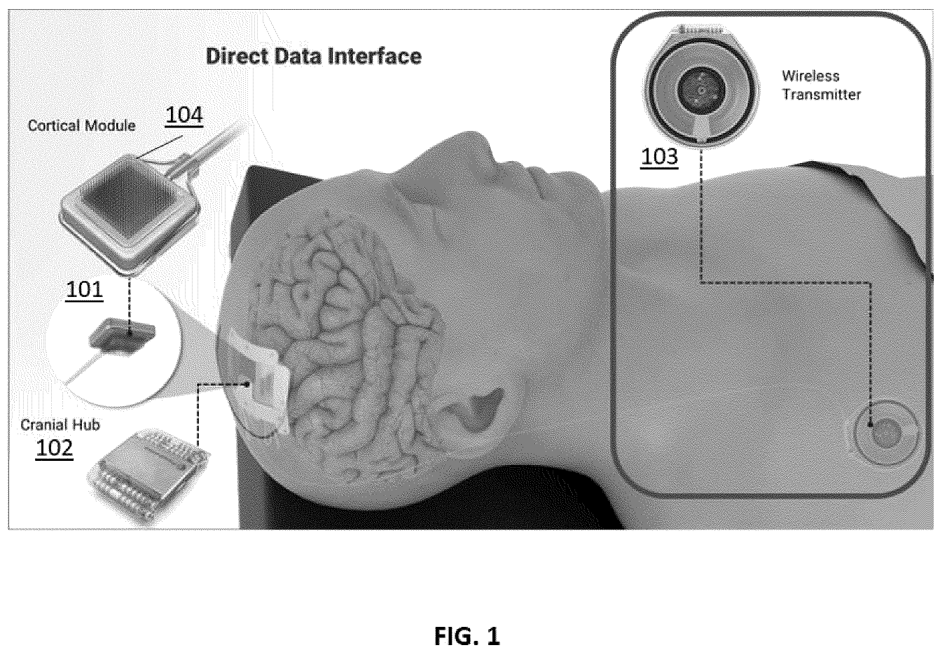

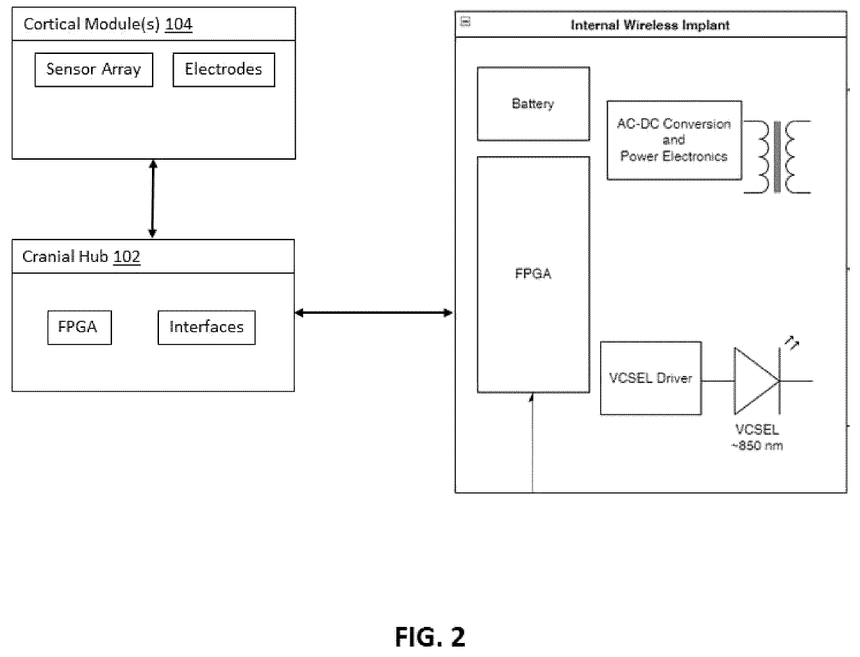

FIG. 2 depicts a system diagram of the DDI system, as shown in FIG. 1, in accordance with some embodiments. In some embodiments, the DDI system may comprise at least one cortical module 101, a cranial hub 102, an internal wireless implant (e.g., wireless transmitter 103), or a combination thereof. In some embodiments, the cortical module comprises a sensor array and an electrode (e.g., wire or probe). In some embodiments, the cortical modules may comprise electrodes but may not comprise detector electronics, wherein the cortical module may be referred to as a passive cortical module. In some embodiments, the cranial hub 102 may comprise an FPGA, interfaces or a combination thereof. In some embodiments, the internal wireless implant 103 may comprise an FPGA, a battery, AC-DC conversion electronics, power electronics, a VCSEL, a VCSEL driver or a combination thereof. The components within the DDI system may be operatively connected to one another via a network or any type of communication link that allows transmission of data from one component to another. The DDI system may be implemented using software, hardware, or a combination of software and hardware in one or more of the above-mentioned components within the system.

### Cranial Hub

Described herein are various embodiments of a direct data interface (DDI) system, the system comprising: at least one cortical module 101 comprising at least one electrode 104; at least one implantable cranial hub (e.g., cranial hub, cranial hub device) 102; and at least one implantable wireless transmitter (e.g., wireless transmitter or internal wireless implant) 103 as depicted in FIG. 1.

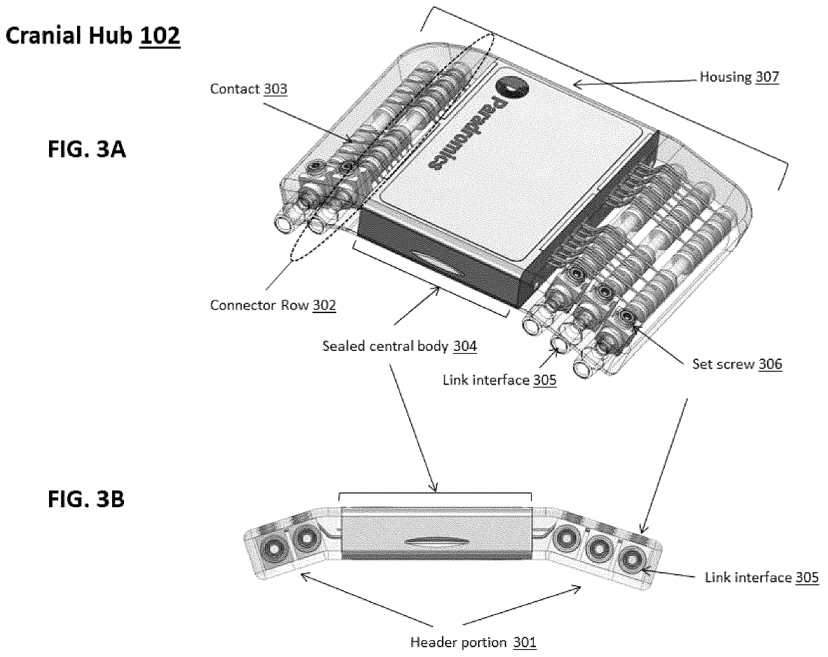

Described herein are various embodiments of the cranial hub 102, where the cranial hub functions as the connecting hub for at least one cortical module 101 and for the at least one wireless transmitter 103 of the DDI system. In some embodiments, the cranial hub connects to at least one cortical module 101. In some embodiments, the cranial hub connects to a plurality of cortical modules 101. In some embodiments, the cranial hub may be implanted in the skull of a subject as depicted in FIG. 1. In some embodiments, the cranial hub may be implanted in or on the skull of a subject as depicted in FIG. 1. In some embodiments, the cranial hub as shown in FIGS. 3A-3B, 4, 5A-5B may be configured to connect to leads 501 that transmit data to and from four cortical modules 101 as well as to and from the at least one wireless transmitter. In some embodiments, the cranial hub may be configured to connect to four cortical modules 101 and to one wireless transmitter via leads 501 to transmit power and/or signals. In some embodiments, the cranial hub connects to one or more cortical modules 101. In some embodiments, the cranial hub connects to four or more cortical modules 101. In some embodiments, the cranial hub connects to 8 or more cortical modules 101.

In some embodiments, the cranial hub 102 connects to at least one other cranial hub.

In some embodiments, the cranial hub may be designed to replace the area of bone removed (e.g., hole in skull of subject or craniotomy) required to access the cortical area of interest of the brain of a subject. In some embodiments, the cranial hub may be implanted next to the area of cortical module 101 implantation. In some embodiments, the cranial hub may be implanted away from the area of cortical module implantation.

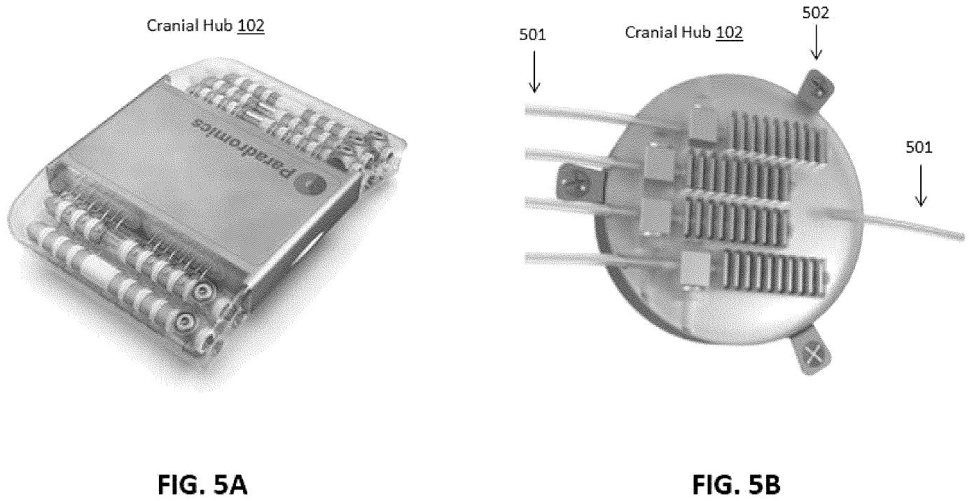

In some embodiments, the cranial hub 102, and flange (e.g., integrated fixture or surface to attach the cranial hub to the skull of a subject) if used, are implanted, or located within a hole (e.g., craniotomy) cut into the skull. In some embodiments, the cranial hub 102 may comprise a flange 502 as depicted in FIG. 5B. In some embodiments, the flange may be configured to secure or attach the hub to the skull of a subject. In some embodiments, the cranial hub may comprise a plurality of flanges.

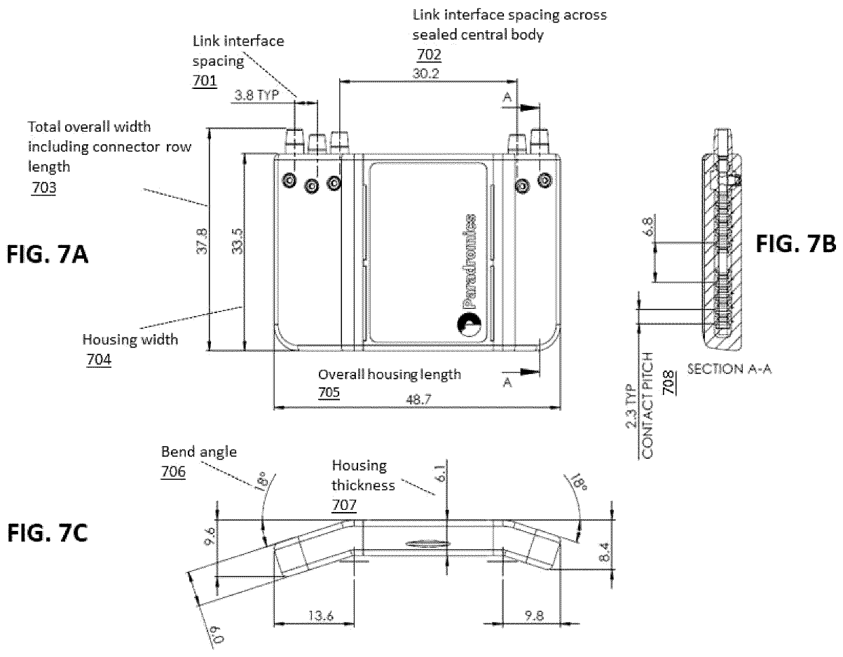

In some embodiments, the cranial hub is designed to closely match the shape of the skull of the subject to limit additional pressure on the brain as well as limit protrusion above the skull. In some embodiments, to facilitate close matching of the curvature of the skull, one or more bends are incorporated into the design of the cranial hub. In some embodiments, the cranial hub may comprise two bends, as shown in the cross-section profile schematics of the cranial hub in FIGS. 3B and 7C.

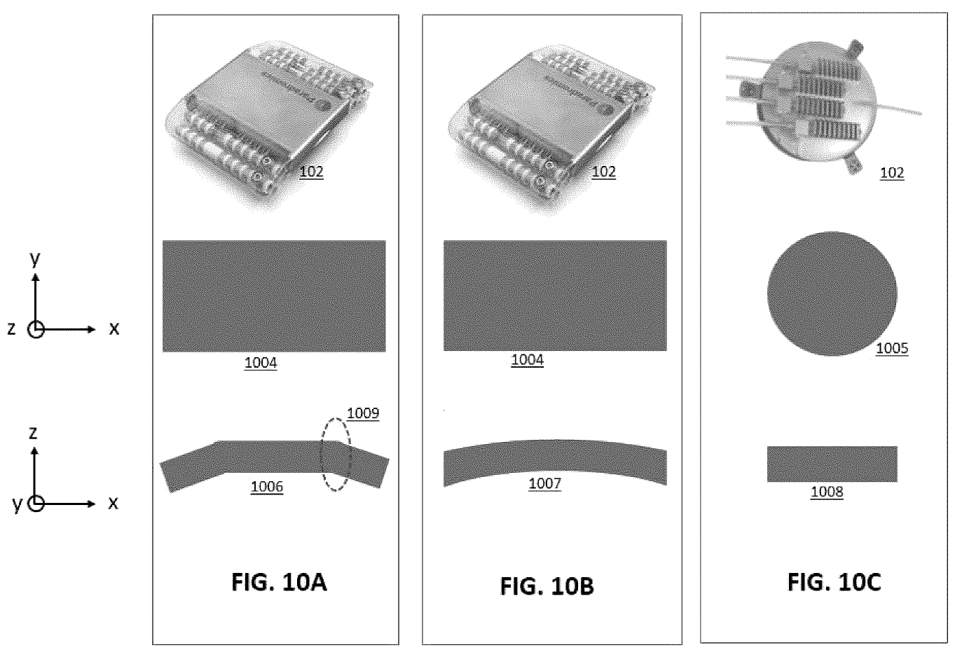

In some embodiments, the cranial hub may comprise a footprint (e.g., xy profile, top-down view, or form factor) as shown in FIGS. 10A-10C. In some embodiments, the footprint may comprise a rectangle 1004, square, or circle 1005 as depicted in FIGS. 10A-10C. In some embodiments, the cranial hub may comprise a side profile (e.g., zx profile). In some embodiments, the side profile may comprise a rectangular central body (e.g., portion) joining at least one other portion at a bend 1009, as depicted in FIG. 10A. In some embodiments, the side profile may comprise a curved side profile 1007, as depicted in FIG. 10B. In some embodiments, the side profile 1008 may comprise a rectangular profile as depicted in FIG. 10C. In some embodiments, the cranial hub comprises a round form factor (e.g., profile 1005). In some examples, ease of bone removal is one advantage of a round form factor. In some embodiments, the cranial hub may comprise a side profile thickness from about 5 mm to about 9 mm. In some embodiments, a cranial hub comprising a round form factor, may comprise a side profile thickness of about 8 mm.

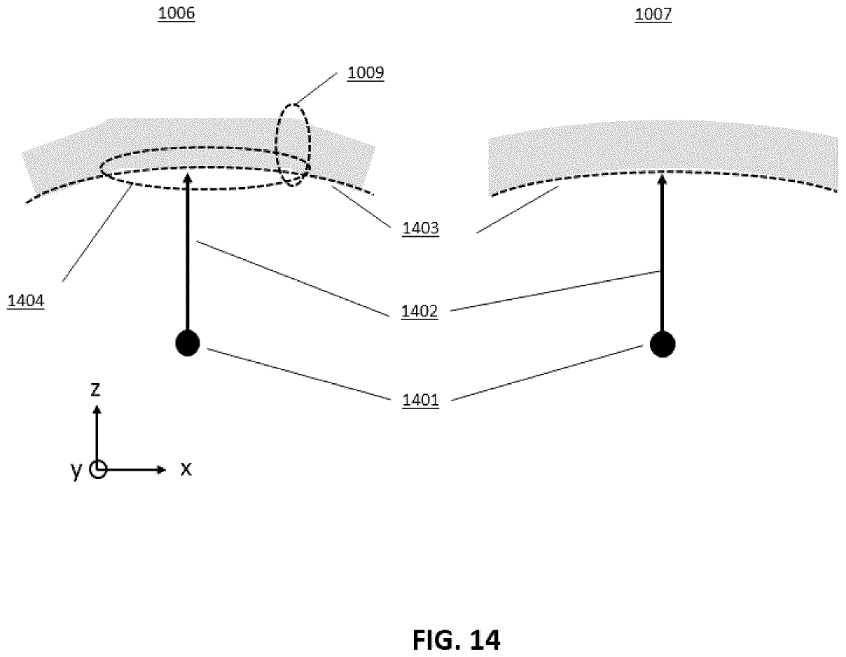

FIGS. 3A-3B depict a three-dimensional view and a side view of the cranial hub 102, in accordance with some embodiments. In some embodiments, the cranial hub may comprise a sealed central body 304 (e.g., a can) as shown in FIGS. 3A-3B. In some embodiments, the sealed central body may be hermetically sealed. In some embodiments, the sealed central body contains electronics. In some embodiments, the cranial hub may comprise a header 301 as shown in FIGS. 3A-3B. In some embodiments, the header may comprise at least one connector row 302 as shown in FIGS. 3A-3B. In some embodiments, the connector row may comprise at least one contact 303, at least one link interface 305, and at least one set screw 306, as shown in FIG. 3A. In some embodiments, the connector row 320 can comprise four connectors arranged on a side of the cranial hub 102 and one link interface 305 arranged on an opposite side of the cranial hub 102. In some embodiments, the housing 307 comprising the four connectors of the connector row 320 and the one link interface 305 can be configured as planar. In some embodiments, the housing 307 comprising the four connectors of the connector row 320 and the one link interface 305 can be configured as curved to form to the skull of the subject. In some embodiments, the cranial hub may comprise a housing 307. In some embodiments, the link interface 305 interfaces with the cortical module 101. In some embodiments, the link interfaces with the wireless transmitter 103. In some embodiments, the housing 307 may comprise a sealed central body 304. In some embodiments, the housing may comprise at least one flange 502. In some embodiments, the header 301 may comprise at least one flange 502. In some embodiments, the header may comprise at least two separate portions as shown in FIGS. 3A-3B. In some embodiments, the housing may contain the electronics and connection elements (e.g., connector rows 302, contacts 303, link interfaces 305). In some embodiments, the housing may comprise the header. In some embodiments, the header may comprise or contain the connector rows 302, the contacts 303 and the link interface 305. In some embodiments, the housing may comprise the header of the device. In some embodiments, the housing may comprise a header (e.g., header portion of cranial hub) 301. In some embodiments, the housing may comprise a curve as depicted in FIG. 14. In some embodiments, the curve may comprise a bended portion 1009 as depicted in FIG. 10A and FIG. 14. In some embodiments, the bended portion 1009 may comprise at least one portion of the housing 307 that is bent or curved relative to a sealed central body (e.g., the can) 304.

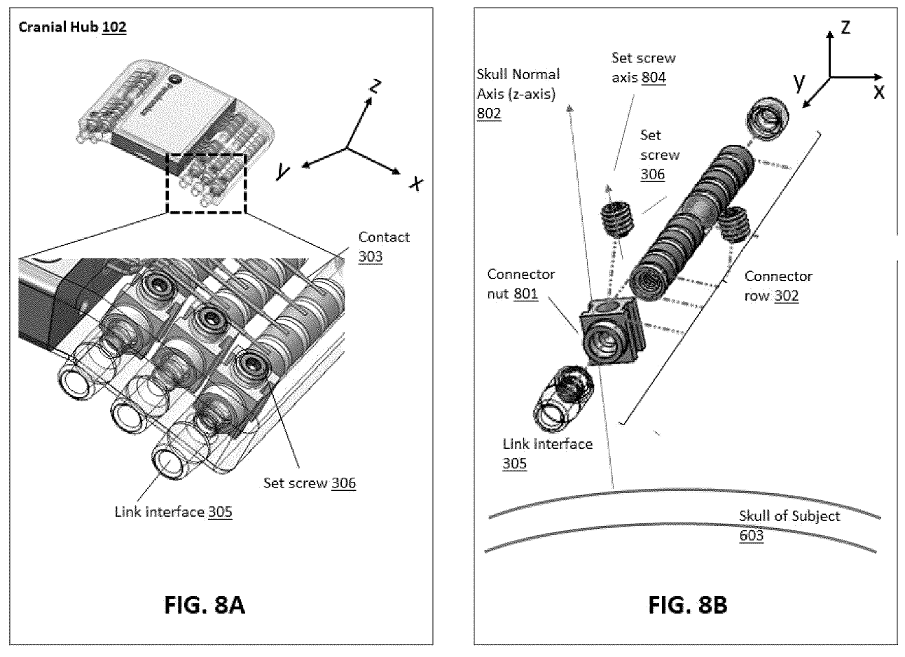

In some embodiments, the leads (e.g., link) 501 may be connected to a series of contacts 303 within the cranial hub via a link interface 305. In such an embodiment, the lead 501 may be mechanically secured with a set screw 306. In some embodiments, the series of connectors rows 302 are offset in a manner such that the set screw 306 may not be obstructed for tightening by inserting the most central lead 501 first and then tightening from the side. In some embodiments, the set screw 306 may be a top accessible set screw. In some embodiments, the top accessible set screw facilitates removal and reattachment of leads 501 while the device may be implanted in the skull of a subject. In some embodiments, following removal and reattachment of a lead 501, a next lead 501 may be inserted and tightened. In some embodiments, the axial offset of the connectors facilitates the ability to access the set screw. FIG. 8A depicts an entire cranial hub and a zoomed in section in accordance with some embodiments. The zoomed in section of FIG. 8A, comprises a link interface 305, a set screw 306, and a contact 303. FIG. 8B shows an exploded-view diagram of the connector row 302, set screw 306, a connector nut 801, and a link interface 305 in relation to a subject's skull 603 in accordance with some embodiments. A skull normal axis (e.g., an axis that is normal to a tangential position on the subject's skull, or z-axis) 802 and a set screw axis 804 are shown in FIG. 8B. In some embodiments the set screw axis 804 may be approximately aligned with the skull normal axis 802. In some embodiments the set screw axis 804 may be about aligned with the skull normal axis 802. In some embodiments the set screw axis 804 may be aligned with the skull normal axis 802. In some embodiments, the set screws 306 may be offset by staggering at a distance (e.g., the staggering distance between set screws) of about 3 mm to about 4 mm. In some embodiments, the staggering distance between set screws may be equal to the link interface spacing 701 as depicted in FIG. 7A. In some examples, the staggering distance between set screws may be about 3.8 mm. In some examples, the staggering distance between set screws may be 3.8 mm.

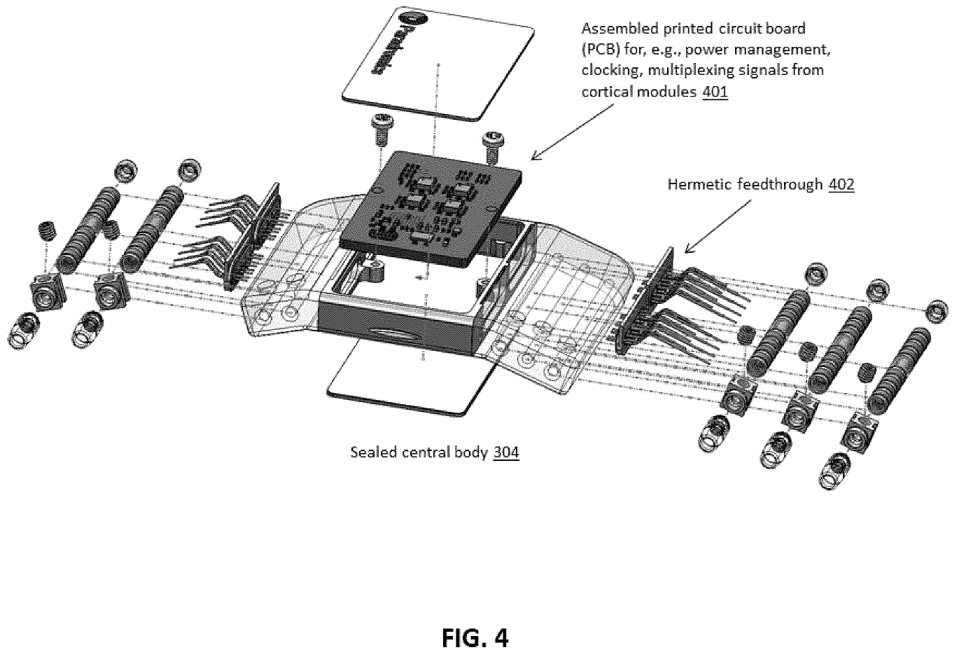

FIG. 4 depicts an exploded view of the cranial hub 102 in accordance with some embodiments. In some embodiments, the sealed central body 304 contains a printed circuit board (PCB) 401. In some embodiments, the PCB 401 may be configured for power management, clocking, multiplexing signals from the cortical modules 101, sending the multiplexed signals to the wireless transmitter 103, or a combination thereof. In some embodiments, as depicted in in FIG. 4, the cranial hub may comprise at least one hermetic feedthrough 402, wherein the hermetic feedthrough may be configured to allow for the contacts 303 in contact with the connector rows 302 to feedthrough the sealed central body 304 and connect to the PCB 401, while maintaining a hermetic seal within the sealed central body 304.

FIG. 14 depicts various embodiments of a cranial hub comprising a housing where the housing may comprise a curve 1403. In some embodiments, the curve may comprise a radius of curvature 1402 and a center of curvature 1401. In some embodiments, as depicted in FIG. 14, the side profile 1007 of the cranial hub housing, may comprise a curve 1403, where the curve matches the side profile of the cranial hub. In some embodiments, as depicted in FIG. 14, the side profile may comprise flat portions 1404 and bended portions 1009, where the curve 1403 may be an approximate fit of the side profile. In some embodiments, the radius of curvature is 80 mm or less. In some embodiments, the radius of curvature is 120 mm or more. In some embodiments, the radius of curvature may be about 80 mm to about 120 mm. In some embodiments, the radius of curvature may be about 100 mm. In some embodiments, the radius of curvature is 100 mm.

Described herein are various embodiments, of a cranial hub 102 configured to reside within the volume of a craniotomy (e.g., hole) of a human skull as depicted in FIG. 1. In some embodiments, the cranial hub may comprise a profile comprising at least one bend to allow for the cranial hub to reside within the craniotomy. FIGS. 7A-7C depict scaled drawings of the cranial hub. FIG. 7A depicts a top-down view scaled drawing of the cranial hub. In some embodiments, the cranial hub may comprise an overall length 705. In some embodiments, the cranial hub overall length 705 may be about 40 mm to about 60 mm. In some embodiments, the cranial hub overall length 705 may be about 48 mm to about 50 mm. In some embodiments, the cranial hub overall length 705 may be 48.7 mm.

In some embodiments, the cranial hub may comprise a housing width (e.g., cranial hub width) 704. In some embodiments, the housing width 704 may be about 30 mm to about 40 mm. In some embodiments, the housing width 704 may be 33.5 mm. In some embodiments, the cranial hub 102 may comprise a total overall width including connector row length 703. In some embodiments, the total overall width including connector row length 703 may be about 35 mm to about 40 mm. In some embodiments, the total overall width including connector row length 703 may be 37.8 mm. In some embodiments, the connector row length overlaps with the housing width. In some embodiments, the cranial hub may comprise a center-to-center spacing of the link interfaces on either side of the sealed central body 702. In some embodiments, the cranial hub may comprise a center-to-center distance between connector rows on either side of the sealed central body 702 of about 25 mm to about 40 mm. In some embodiments, the cranial hub may comprise a center-to-center distance between connector rows across the sealed central body of 30.2 mm. In some embodiments, the cranial hub may comprise a contact pitch 708 (e.g., center-to-center spacing of contacts) along the connector row 302. In some embodiments, the contact pitch 708 may be 2.3 mm, as shown in FIG. 7B. FIG. 7C depicts a scaled drawing of the side profile of the cranial hub in accordance with some embodiments.

In some embodiments, the housing thickness 707 may be about 5 to about 8 mm. In some embodiments, the housing thickness 707 may be about 6 mm. In some embodiments, the housing thickness 707 may be 6.0 mm as depicted in FIG. 7C. In some embodiments, the housing thickness 707 may be 6.1 mm. In some embodiments, the housing thickness as measured at the sealed central body 304, may be 6.1 mm and the housing thickness 707 at the edge of the header 301 may be 6.0 mm.

In some embodiments, the cranial hub may comprise a profile as depicted in FIG. 7C. In some embodiments, the profile may comprise at least one bend. In some embodiments, the profile may comprise 2 bends. In some embodiments, as depicted in FIG. 7C the bend may comprise a bend angle 706. In some embodiments, the bend angle 706 may be about 15° to about 20°. In some embodiments, the bend angle 706 may be about 18°. In some embodiments, the bend angle 706 is 18°. In some embodiments, the angle between a horizontal line running through the plane of the top surface of the sealed central body 304 (e.g., top of the housing containing the sealed central body) relative to the top surface of the header defines the bend angle as depicted in FIG. 7C.

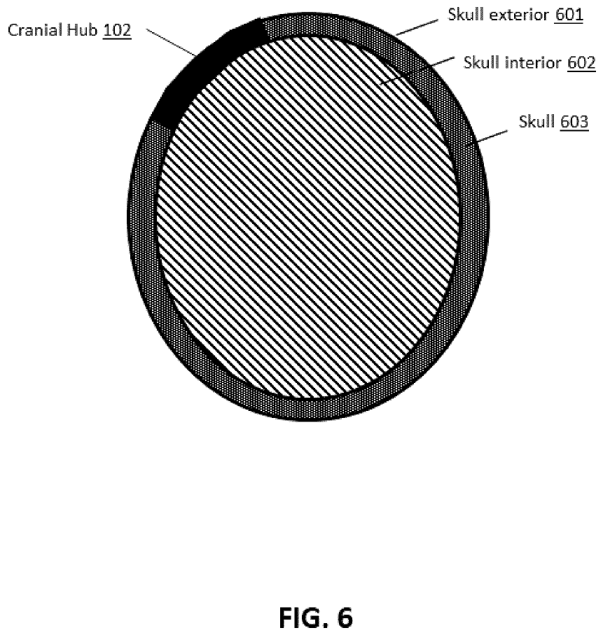

Described herein are various embodiments of a cranial hub 102 designed to reside within the profile of a human skull 603 as depicted in FIG. 6. In some embodiments, values of at least the dimensions of the bend angle 706 and of the housing thickness were determined to allow for the cranial hub to reside within the thickness of the skull with minimal to no protrusion above the outer surface of the skull (e.g., exterior of the skull) 601. In some embodiments, the implanted cranial hub is in contact with the interior of the skull 602. In some embodiments, the radius of curvature 1402 of a cranial hub may be determined by computed tomography (e.g., CT) scan data. In some embodiments, the cranial hub may be designed to replace the region of bone removed from the skull (e.g., craniotomy) that is required to access the cortical area of interest of the brain. In some embodiments, the cranial hub may be implanted next to the region of cortical module implantation. In some embodiments, the cranial hub may be implanted away from the region of cortical module implantation.

In some embodiments, as depicted in FIG. 5A, the cranial hub may comprise a rectangular shaped top-down profile (e.g., footprint or form-factor). In some embodiments, the cranial hub may comprise a rounded, or circular shaped, top-down view profile (e.g., footprint) as depicted in FIG. 5B.

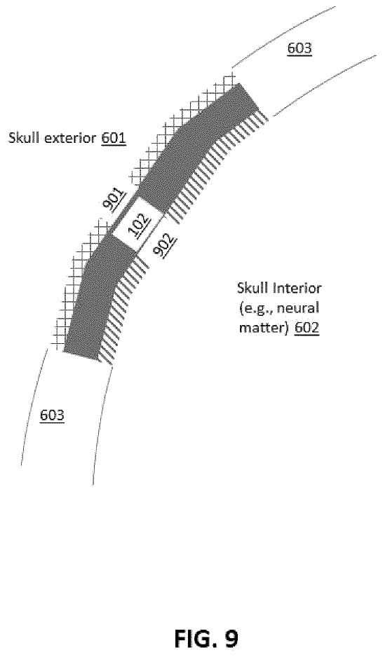

An implantable cranial hub 102 comprising layers is depicted in FIG. 9, in accordance with some embodiments. In some embodiments, the cranial hub may be implanted in a skull of a subject 603. In some embodiments, the implantable cranial hub may comprise a housing 307, comprising at one least layer, wherein the layer may be configured to minimize heat transfer to the interior of the skull (e.g., neural matter) of a subject 602 and/or maximize heat loss to outside the skull of a subject 601.

In some embodiments, where the layer is located between the implantable cranial hub 102 and the interior of the skull 602 and wherein the layer may be configured to block heat transfer from the implantable cranial hub to the interior of the skull 602, the layer may be referred to as a heat blocking layer 902, as depicted in FIG. 9. In some embodiments, the heat blocking layer 902 may comprise a heat blocking material. In some embodiments, the heat blocking material comprises metal, ceramic or a polymer. In some embodiments, the metal comprises titanium. In some embodiments, the heat blocking material comprises a polyethylene polymer or a PEEK polymer. In some embodiments, the heat blocking material comprises an alumina ceramic or a zirconia ceramic. In some embodiments, the heat blocking material may comprise silicon carbide. In some embodiments, the heat blocking material may comprise epoxy.

In some embodiments, the layer is located between the implantable cranial hub and the exterior of the skull 601 and where the layer is configured to transfer heat from the implantable cranial hub to the exterior of the skull, the layer may be referred to as a heat transfer layer 901, as depicted in FIG. 9. In some cases, the layer is designed to maximize heat dissipation to the blood supply thereby reducing overall temperature of the device and the surrounding tissue. In some embodiments, the heat transfer layer may comprise a heat transfer material. In some embodiments, the heat transfer material may be a metal such as titanium metal or stainless steel. In some embodiments, the heat transfer material may be a structured device comprising metals such as titanium, platinum, or gold. In some embodiments, the heat transfer material may comprise titanium, platinum, or gold.

In some embodiments, the layer is configured to maximize heat loss to a vascularization of one or more tissues surrounding the cranial hub. In some embodiments, the layer is configured to utilize one or more growth factors to promote a growth of one or more tissues surrounding the cranial hub. In some embodiments, the one or more growth factors comprises a surface texture of the layer. In some embodiments, the layer is configured to be in close proximity to an existing high vascularization.

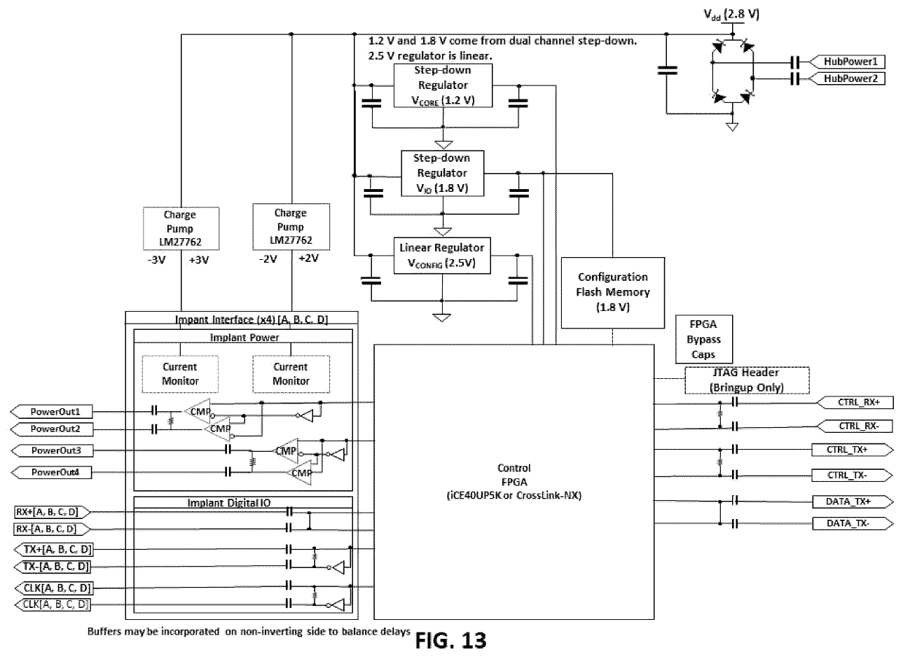

Described herein are various embodiments of cranial hub interfaces, as depicted in FIG. 13. In some embodiments, the cranial hub interfaces may comprise physical interfaces. In some embodiments physical interfaces may comprise Bal Seal® connectors configured to allow connections to custom leads.

In some embodiments, one of the cranial hub interfaces may comprise a wireless transmitter (e.g., transceiver implant) 103 electrical interface. In some embodiments, such interfaces may be configured for lower power and increased electromagnetic compatibility (EMC). In some embodiments, the wireless transmitter electrical interfaces may be configured to support protocols comprising differential asynchronous slow control or command data from the wireless transmitter. In some embodiments, such protocols comprise 8b10b, Manchester, or other DC-balanced encoding. In some embodiments, the wireless transmitter electrical interfaces may be configured to support protocols comprising differential fast neural data to the wireless transmitter with DC balanced encoding (e.g, 8b10b). In some examples, the wireless transmitter electrical interfaces may be configured to support protocols comprising differential power from a wireless transmitter.

Described herein are various embodiments of the wireless transmitter (e.g., transceiver implant). Electrical interfaces may be configured to support protocols comprising higher power, or a fully synchronous system, which could result in higher EM emissions. In some embodiments, the wireless transmitter electrical interfaces may be configured to support protocols comprising differential clock from the wireless transmitter. In some embodiments, the wireless transmitter electrical interfaces may be configured to support protocols comprising differential slow control or command data from the wireless transmitter. In some embodiments, the wireless transmitter electrical interfaces may be configured to support protocols comprising differential fast neural data and command responses to the wireless transmitter, 8b10b encoded. In some embodiments, the wireless transmitter electrical interfaces may be configured to support protocols comprising differential power from the wireless transmitter.

Described herein are various embodiments of cranial hub interfaces that may comprise cortical module interfaces. In some embodiments, cortical module facing interfaces may be configured to support protocols comprising differential clock out to each cortical module. In some embodiments, cortical module facing interfaces comprise differential data out (e.g., control/commands) to each cortical module on an 8b10b encoded link. In some embodiments, cortical module facing interfaces comprise differential data in (e.g., neural data, command responses) from each cortical module on an 8b10b encoded link. In some embodiments, cortical module facing interfaces comprise differential power out to cortical module.

Described here are various embodiments of implantable cranial hub devices comprising: a housing, the housing comprising a curve, the curve comprising a radius of curvature; and a plurality of connector rows, wherein the plurality of connector rows may be configured to operably couple to a plurality of implantable cortical modules (e.g., cortical module); and electronic circuitry configured to (1) aggregate a plurality of signals received from the plurality of implantable cortical modules and (2) transmit the aggregated plurality of signals to at least one wireless transmitter. In some embodiments, the radius of curvature is 80 mm or less. In some embodiments, the radius of curvature is 120 mm or more. In some embodiments, the radius of curvature may be about 80 mm to about 120 mm. In some embodiments, the radius of curvature may be about 100 mm. In some embodiments, the curve may comprise at least one portion that may be bent and at least one portion that may be flat. In some embodiments, the at least one portion that may be bent may comprise a bend angle, θ. In some embodiments, the bend angle, θ, is 15° or less. In some embodiments, the bend angle, θ, is 20° or more. In some embodiments, the bend angle, θ, may be about 15° to about 20°. In some embodiments, the bend angle, θ, may be about 18°. In some embodiments, the device further may comprise a flange. In some embodiments, the flange may be configured to permit the cranial hub to be mounted to a skull of a subject.

In some embodiments, the cranial hub 102 may comprise a wireless transmitter 103.

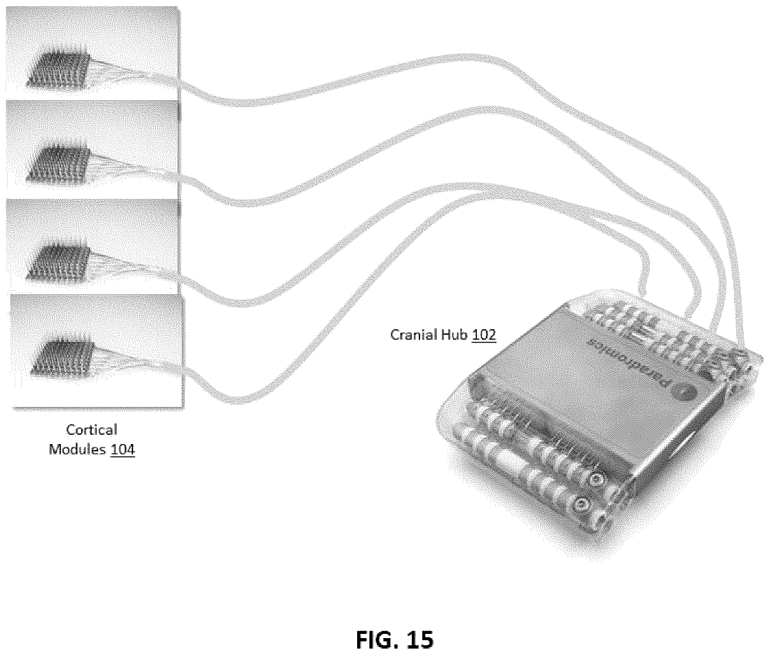

FIG. 15 depicts multiple passive cortical modules linked to a cranial hub in accordance with some embodiments. In such an embodiment, sensor electronics (e.g., CMOS-based sensor array) are housed within the cranial hub and not the cortical module. In certain aspects, where passive cortical modules are connected to the cranial hub, each wire (e.g., electrode) 104 of the passive cortical module is individually connected to the cranial hub.

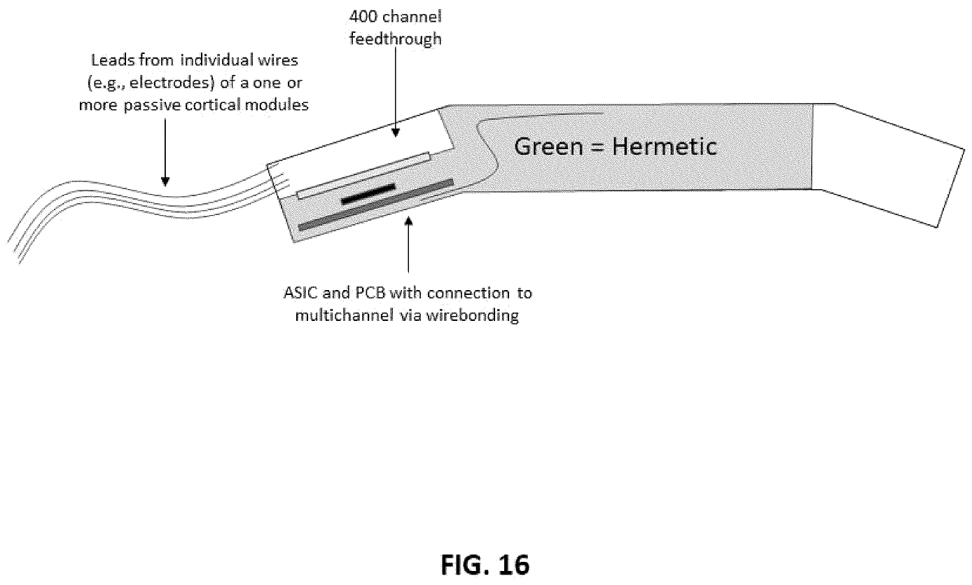

FIG. 16 depicts a cranial hub 102 having a hermetic feedthrough configured to connect to individual wires (e.g., electrodes) 104 from multiple passive cortical modules in accordance with some embodiments. In such embodiments, leads from individual wires (e.g., electrodes) of one or more passive cortical modules are separately connected to the cranial hub, where the signal from each electrode is aggregated with the signal from other electrodes of the same passive cortical module and/or from other passive cortical modules. In some embodiments, where at least one passive cortical module is connected, the cranial hub may comprise a multichannel feedthrough. In some embodiments, the multichannel feedthrough may be configured to allow for a hermetic seal of the cranial hub. In some embodiments, the multichannel feedthrough may comprise 1 to 1000 channels. In some embodiments, the multichannel feedthrough may comprise 1000, or greater, channels. In some embodiments, the multichannel feedthrough may comprise 400 channels.

### Cranial Hub Frames or Flanges

In some cases, the cranial hub 102 may be attached to the skull of the subject. In some cases, the attaching can include use of a frame with one or more flanges that can be configured to attach the cranial hub 102 to the skull of the subject. In some cases, the frame can also be configured to attach to or be formed into (e.g., molded into) the cranial hub 102. In some cases, the frame can be configured to attach to or be formed into a superior side or an anterior side of the cranial hub 102. In some cases, the frame can include a first part configured to attach to or be formed into to the superior side of the cranial hub 102 and a second part configured to attach to or be formed into the anterior side of the cranial hub 102. In some cases, the one or more flanges can be attached to or formed into the frame. In some cases, the one or more flanges can be attached to or formed into the cranial hub 102. In some cases, the one or more flanges can be attached to or formed into the frame, the cranial hub 102, or a combination thereof. In some cases, the one or more flanges contact or attach to the skull of the subject to secure the cranial hub 102, the frame, or both to the skull of the subject. In some cases, the frame or the one or more flanges may be oversized but configured to be trimmed to fit or form to the skull of the subject.

In some cases, the frame can include one or more flanges. In some cases, the frame can include one or more edges or sides. In some cases, the one or more flanges can be attached to or be formed into the one or more edges or sides of the frame. In some cases, the frame can include a central region. In some cases, the central region of the frame can include one or more edges or sides. In some cases, the central region of the frame can be a shape including circular, semicircular, or elliptical. In some cases, the one or more flanges can be attached to or be formed into the one or more edges or sides of the central region of the frame. In some cases, the one or more flanges can be attached to or be formed into the one or more edges or sides of the cranial hub 102.

In some cases, the one or more edges or sides of the frame, the central region of the frame, or the cranial hub 102 can be a same length, width, or thickness. In some cases, each of the one or more edges or sides of the frame, the central region, or the cranial hub 102 can be a different length, width, or thickness. In some cases, a length of the one or more edges or sides of the frame, central region, or the cranial hub 102 can include a range of about 0.5 inches to about 5 inches. In some cases, a width of the one or more edges or sides of the frame, central region, or the cranial hub 102 can include a range of about 0.5 inches to about 5 inches. In some cases, a thickness of the one or more edges or sides of the frame, the central region, or the cranial hub 102 can include a range of about 0.1 inches to about 0.5 inches.

In some cases, the one or more flanges can be spaced equally around the one or more edges or sides of the frame. In some cases, the one or more flanges can be spaced equally around the one or more edges or sides of the central region of the frame. In some cases, the one or more flanges can be spaced equally around the one or more edges or sides of the cranial hub 102. In some cases, each of the one or more flanges can be spaced with a different centerline spacing between the one or more flanges around the one or more edges or sides of the frame. In some cases, each of the one or more flanges can be spaced with a different centerline spacing between the one or more flanges around the one or more edges or sides of the central region of the frame. In some cases, each of the one or more flanges can be spaced with a different centerline spacing between the one or more flanges around the one or more edges or sides of the cranial hub 102. In some cases, the centerline spacing between the one or more flanges can include a range of about 0.2 inches to about 2 inches.

In some cases, each of the one or more flanges can be attached to or be formed into the one or more edges or sides of the frame at a same angle, θ, relative to the one or more edges or sides of the frame. In some cases, each of the one or more flanges can be attached to or be formed into the one or more edges or sides of the central region of the frame at a same angle, θ, relative to the one or more edges or sides of the central region of the frame. In some cases, each of the one or more flanges can be attached to or be formed into the one or more edges or sides of the central region of the frame at a same angle, θ, relative to the one or more edges or sides of the cranial hub 102. In some cases, each of the one or more flanges can be attached to or be formed into the one or more edges of the frame at different angles, e.g., θ₁, θ₂, θ₃, and, so on, relative to the one or more edges of the frame. In some cases, each of the one or more flanges can be attached to or be formed into the one or more edges of the central region at different angles, e.g., θ₁, θ₂, θ₃, and, so on, relative to the one or more edges of the central region. In some cases, each of the one or more flanges can be attached to or be formed into the one or more edges of the cranial hub 102 at different angles, e.g., θ₁, θ₂, θ₃, and, so on, relative to the one or more edges of the cranial hub 102. In some cases, any one the angles, θ₁, θ₂, θ₃, and, so on, can include a range of about 0 degrees to 180 degrees.

In some cases, the one or more flanges can be of a same length, width, and thickness. In some cases, each of the one or more flanges can be of a different length, width, or thickness. In some cases, a length of each of the one of the one or more flanges can include a range of about 0.5 inches to about 5 inches. In some cases, a length of any one of the one or more flanges can include a range of about 0.5 inches to about 5 inches. In some cases, a width of each of the one or more flanges can include a range of about 0.5 inches to about 5 inches. In some cases, a width of any one of the one or more flanges can include a range of about 0.5 inches to about 5 inches. In some cases, a thickness of each of the one or more flanges can include a range of about 0.1 inches to about 0.5 inches. In some cases, a thickness of any one of the one or more flanges can include a range of about 0.1 inches to about 1 inch.

In some cases, the frame can include one or more openings (e.g., cutouts or windows) configured to allow a user to view some regions or all regions of the cranial hub 102. In some cases, the windows are configured to allow a user to access one or more regions of the cranial hub 102 via the windows, e.g., repairing or troubleshooting the cranial hub 102. In some cases, a shape of any one of the one or more windows can be circular, semicircular, square, rectangular, or elliptical. In some cases, the one or more windows can be spaced equally around the frame. In some cases, the one or more windows can be spaced with a different spacing between the one or more windows around the frame. In some cases, a centerline spacing between the one or more windows can include a range of about 0.2 inches to about 2 inches. In some cases, the one or more windows can be configured as an array of windows. In some cases, the array can include an array of 1, 2, 3, or more windows. In some cases, the array can be configured as a 1 by n array, an n by n array, or an n by m to accommodate a number of the one or more windows.

In some cases, the one or more windows can be of an equal length, width, and thickness. In some cases, each of the one or more windows can be of a different length, width, or thickness. In some cases, a length of any one of the one or more windows can include a range of about 0.1 inches to about 0.5 inches. In some cases, a width of any one of the one or more windows can include a range of about 0.1 inches to about 0.5 inches. In some cases, a thickness of any one of the one or more windows can include a thickness that extends through the entire thickness of the frame.

In some cases, the frame can include a means for attaching the frame to the skull of the subject. In some cases, the means can include fasteners (e.g., screws), clips, snap fits, or slip fits. In some cases, the one or more flanges of the frame or the cranial hub 102 can include one or more holes configured to accept one or more fasteners (e.g., screws) for attaching the cranial hub 102 to the skull of the subject. In some cases, the frame or the cranial hub 102 can include one or more shims to position the cranial hub 102 at an optimal location in the skull of the subject, e.g., sit in a lower or higher profile relative to the skull. In some cases, the combination of the frame and the cranial hub 102 can be configured to attach to the skull of the subject without use of the one or more fasteners. In some cases, the configuring can include forming or shaping the frame to the skull of the subject to secure the cranial hub 102 to the skull through a slip fit. In some cases, the frame or the flanges can be fabricated from a formable or malleable material such as polyethylene, titanium, epoxy, or a combination thereof. In some cases, the frame or the one or more flanges can be formed or shaped before, during, or after surgery to fit the size or shape of the skull of the subject. In some cases, the frame or the flanges can be custom manufactured from preoperative CT or MRI data to match or form to the patient's skull anatomy.

In some cases, the frame can include a means for attaching the frame to the cranial hub 102. In some cases, the means for attaching the frame to the cranial hub 102 can include fasteners (e.g., screws or tensioning screws), fastener tabs, welding (e.g., laser welding or tack welding), clips, snap fits, slip fits, adhesives, and the like. In some cases, the cranial hub 102 can include one or more welding tabs configured to allow the frame to be welded to the cranial hub 102. In some cases, the frame can be attached to or formed into the superior side, the anterior side, or a combination thereof of the cranial hub 102.

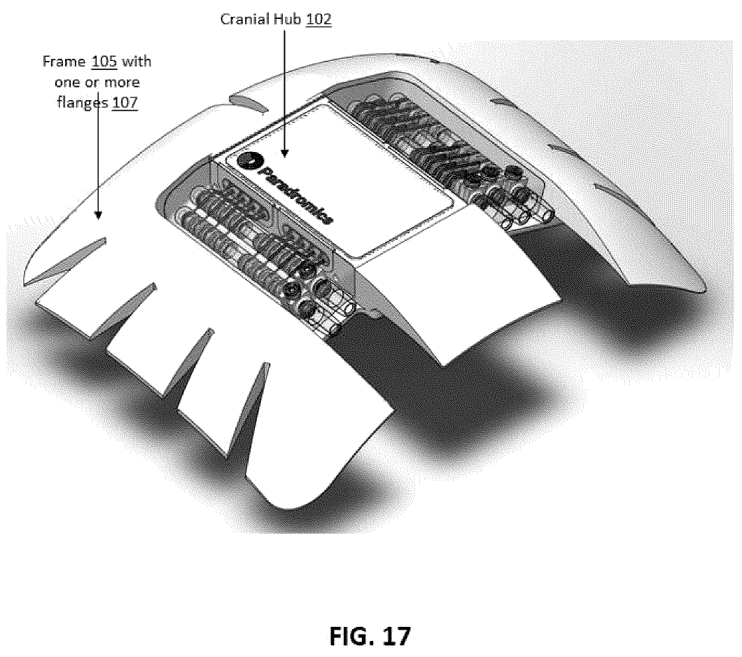

FIG. 17 generally depicts a cranial hub 102 including a frame 105 with one or more flanges 107 for attaching the cranial hub 102 to a skull of a subject. In some cases, the frame 105 is configured to attach to the superior side or the anterior side of the cranial hub 102. In some cases, the frame 105 is configured to cradle the cranial hub 102. In some cases, the attaching can include a snap fit using one or more fastening tabs 113 (not shown). In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. In some cases, the frame 105 can be attached to the cranial hub 102 by one or more fastening tabs 113 (not shown). In some cases, the frame 105 can include ten flanges 107 of a same or different shape or length. In some cases, four flanges 107 can be located on each of two opposite edges or sides of the central region of the frame 105. In some cases, two flanges 107 can be located on a back side of the central region of the frame 105. In some cases, each flange 107 can include one or more holes (not shown) for inserting one or more fasteners (e.g., screws) to attach the cranial hub 102 to the skull of a subject via the holes (not shown) of the flanges 107. In some cases, the frame 105 can be made of a material such as polyethylene.

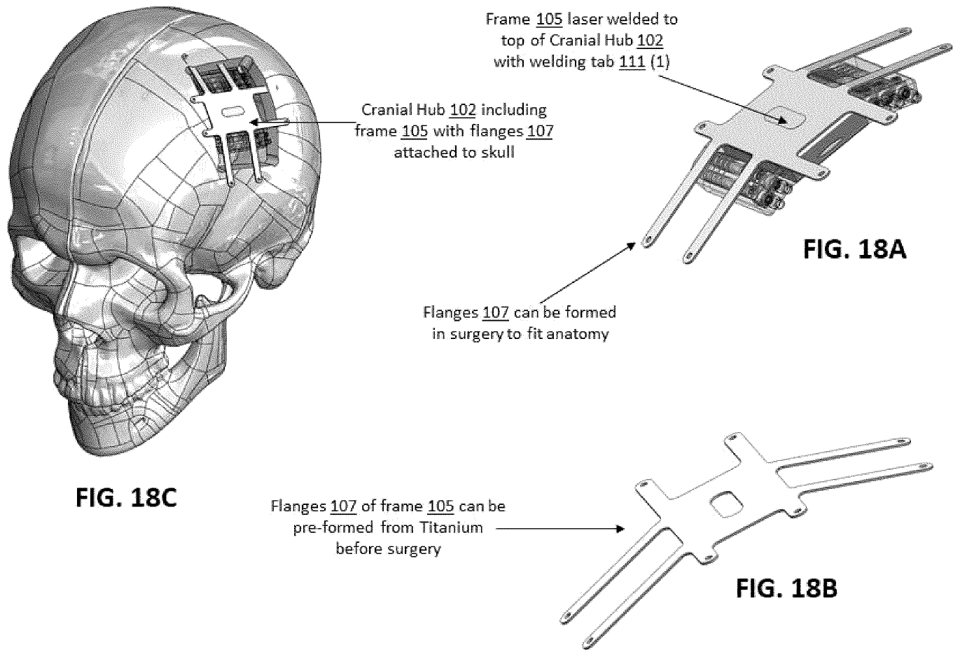

FIGS. 18A-18C depict a cranial hub 102 including a frame 105 with one or more flanges 107 configured to attach the cranial hub 102 to a skull of a subject. In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. Illustrated in FIG. 18A is a superior view of the frame 105 that can be attached to or formed into the superior side of the cranial hub 102. In some cases, the frame 105 can be attached to the cranial hub 102 by welding (e.g., laser welding) the frame 105 to one or more welding tabs 111 located on the cranial hub 102. In some cases, the one or more welding tabs 111 (e.g., 1 welding tab) can be inserted into one or more openings (e.g., 1 opening) of the frame 105 before welding. In some cases, the frame 105 can include eight flanges 107 of different lengths with two flanges 107 on each edge or side of the central region of the frame 105. In some cases, each flange 107 can include a hole for inserting a fastener (e.g., a screw) to attach the cranial hub 102 to the skull of a subject via the holes of the flanges 107. FIG. 18B depicts a superior view of the frame 105, the flanges 107, and the hole for welding the frame 105 to the cranial hub 102. FIG. 18C depicts the cranial hub 102 inserted into the skull of a human subject and secured to the human skull via the frame 105 and the flanges 107. In some cases, the frame 105 can be made of a material such as Titanium.

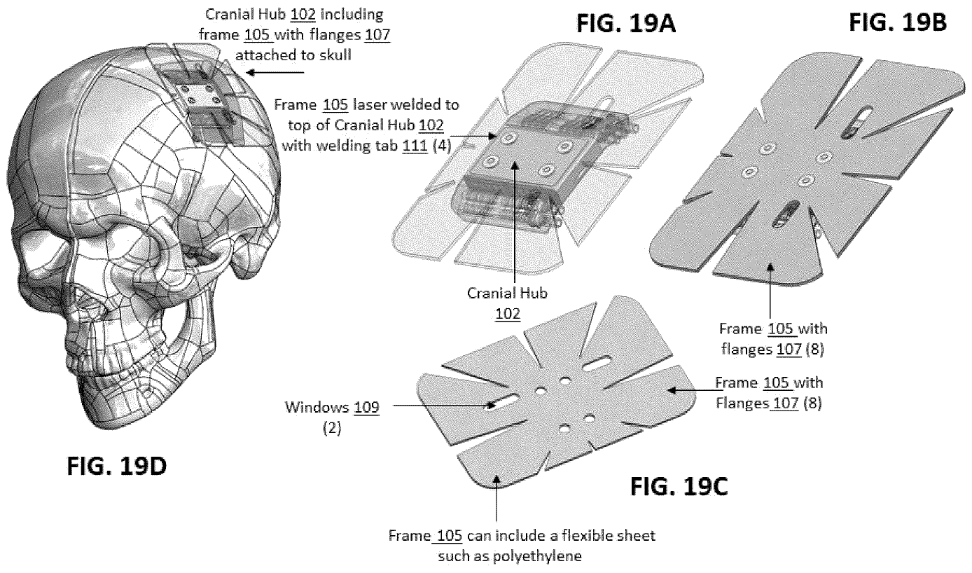

FIGS. 19A-19D depict a cranial hub 102 including a flexible frame 105 with one or more flanges 107 configured to attach the cranial hub 102 to a skull of a subject. In some cases, the frame 105 or the flanges 107 can be made from a shapeable or formable material such as polyethylene so that the frame 105 can form to the skull of the subject. In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. Illustrated in FIG. 19A is a superior view of the frame 105 that can be attached to or formed into the superior side of the cranial hub 102. In some cases, the frame 105 can be attached to the cranial hub 102 by welding (e.g., laser welding) the frame 105 to one or more welding tabs 111 (e.g., 4 welding tabs) located on the cranial hub 102. In some cases, the one or more welding tabs 111 can be inserted into one or more openings (e.g., 4 openings) of the frame 105 before welding. In some cases, the frame 105 can include eight flanges 107 of different shapes or lengths with two flanges 107 on each edge or side of the central region of the frame 105. In some cases, each flange 107 can include a hole (not shown for clarity) for inserting a fastener (e.g., a screw) to attach the cranial hub 102 to the skull of a subject via the holes of the flanges 107. In some cases, the frame 105 can be made of a material such as polyethylene. FIG. 19B depicts another superior view of the cranial hub 102 including the flexible frame 105 with the one or more flanges 107 that can include formable flanges for attaching the cranial hub 102 to a skull of a subject. FIG. 19C depicts the anterior view of the flexible frame 105 of FIG. 19A or FIG. 19B with the one or more flanges 107 that can include formable flanges for attaching the cranial hub 102 to a skull of a subject. In some cases, the frame 105 can include one or more windows 109 (e.g., 2 or 4). FIG. 19D depicts the cranial hub 102 inserted into the skull of a human subject and secured to the human skull via the flexible frame 105 and the flanges 107. In some cases, the frame 105 can be made of a material such as Titanium.

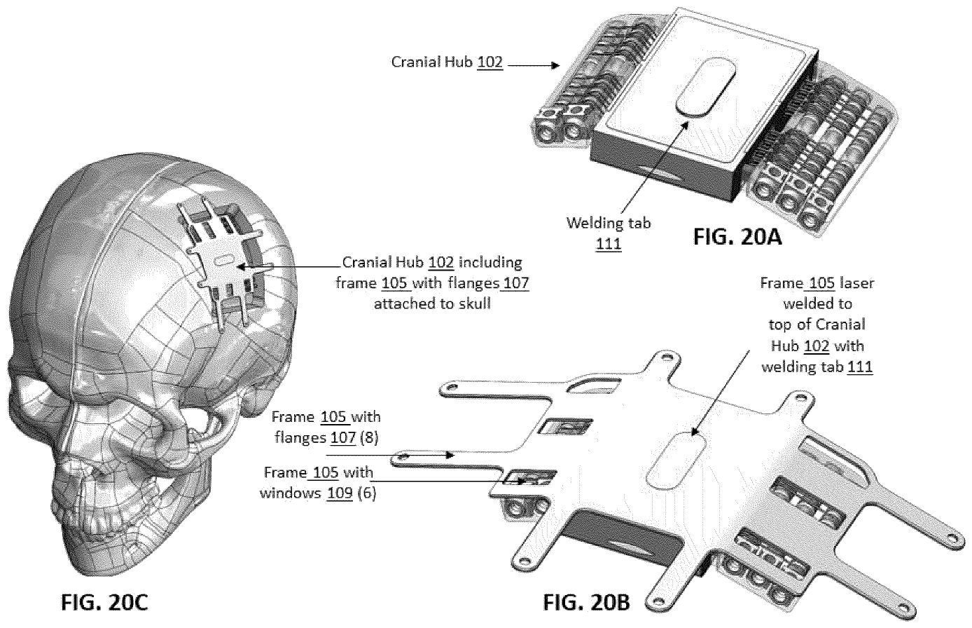

FIGS. 20A-20C depict a cranial hub 102 including a flexible frame 105 with one or more flanges 107 configured to attach the cranial hub 102 to a skull of a subject. In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. Illustrated in FIG. 20A is a superior view of the cranial hub 102 with one or more welding tabs 111 (e.g., 1 welding tab). FIG. 20B depicts a superior view of the frame 105, the flanges 107, and the hole for welding the frame 105 to the cranial hub 102. In some cases, the frame 105 can be attached to the cranial hub 102 by welding (e.g., laser welding) the frame 105 to one or more welding tabs 111 (e.g., 1 welding tab) located on the cranial hub 102. In some cases, the one or more welding tabs 111 (e.g., 1 welding tab) can be inserted into one or more openings (e.g., 1 opening) of the frame 105 before welding. In some cases, the frame 105 can include eight flanges 107 of different lengths with two flanges 107 on each edge or side of the central region of the frame 105. In some cases, each flange 107 can include a hole for inserting a fastener (e.g., a screw) to attach the cranial hub 102 to the skull of a subject via the holes of the flanges 107. In some cases, the frame 105 can include one or more windows 109 (e.g., 6 windows) configured to view some or all regions of the cranial hub 102 from above the frame 105. FIG. 20C depicts the cranial hub 102 inserted into the skull of a human subject and secured to the human skull via the frame 105 and the flanges 107. In some cases, the frame 105 can be made of a material such as Titanium.

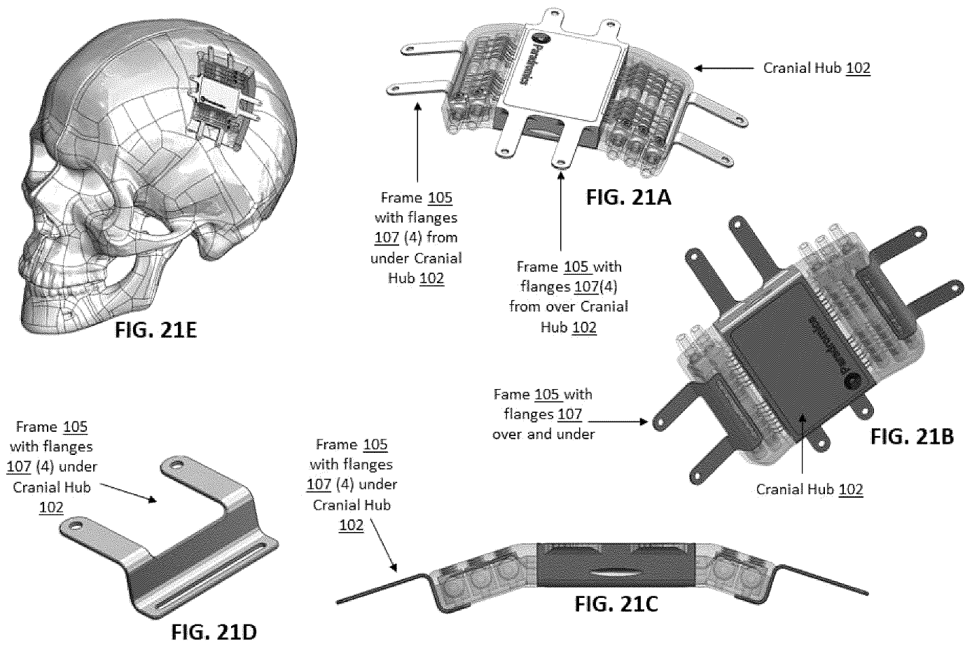

FIGS. 21A-21E depict a cranial hub 102 including a frame 105 with one or more flanges 107 that can include flanges 107 on the superior side and the anterior side of the cranial hub 102 configured to attach the cranial hub 102 to a skull of a subject, in accordance with some embodiments. In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. Illustrated in FIG. 21A is a superior view of the cranial hub 102 with one or more flanges 107. In some cases, the frame 105 can include a first part on the superior side of the cranial hub 102 and a second part and a third part on the anterior side of the cranial 102. In some cases, the first part of the frame 105 can be attached to or formed into the cranial hub 102. In some cases, the second and third parts of the frame 105 can be attached to the anterior side of the cranial hub 102 and partially or completely wrap around to the superior side of the cranial hub 102. In some cases, the second part of the frame 105 is on an end and the third part of the frame 105 is on an opposite end of the cranial hub 102. In some cases, the second and third parts of the frame 105 can attach to the cranial hub 102 by a snap fit or a slip fit. In some cases, the frame 105 can include eight flanges 107 of different lengths with four flanges 107 on the first part of the frame 105, two flanges 107 on the second part of the frame 105, and two flanges 107 on the third part of the frame 105. In some cases, each flange 107 can include a hole for inserting a fastener (e.g., a screw) to attach the cranial hub 102 to the skull of a subject via the holes of the flanges 107. In some cases, the frame 105 can be made of a material such as Titanium. FIG. 21B depicts the anterior view of the cranial hub 102 of FIG. 21A, the frame 105, and the flanges 107. The anterior view also depicts the second and third parts of the frame 105 on the anterior side of the cranial hub 102. FIG. 21C depicts a side view of the cranial hub 102 of FIG. 21A, the frame 105, and the flanges 107. The side view also depicts the second and third parts of the frame 105. FIG. 21D depicts an anterior view of the first part or the second part of the frame 105. FIG. 21E depicts the cranial hub 102 inserted into the skull of a human subject and secured to the human skull via the frame 105 and the flanges 107. In some cases, the frame 105 can be made of a material such as Titanium.

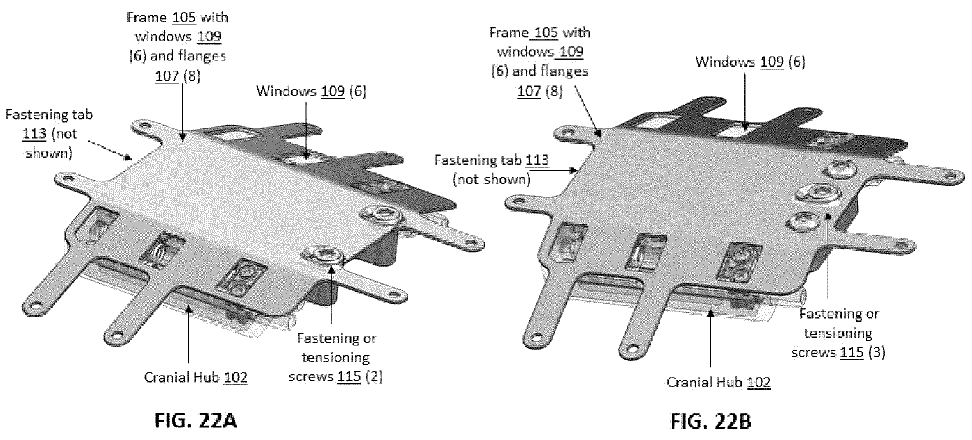

FIGS. 22A-22B depict a cranial hub 102 including a frame 105 with one or more viewing windows 109 and one or more flanges 107 configured to attach the cranial hub 102 to a skull of a subject. In some cases, the frame 105 is configured to attach to the superior side of cranial hub 102 with fasteners or tensioning screws and one or more fastening tabs 113, in accordance with some embodiments. In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. FIG. 22A depicts a superior view of the cranial hub 102 including the frame 105, the one or more flanges 107 (e.g., 6 flanges), one or more windows 109 (e.g., 6 windows), one or more fastening screws 113 (e.g., 2 screws), and one or more fastening tabs 113 (e.g., 1 tab, not shown for clarity). In some cases, the frame 105 can be attached to the cranial hub 102 by a combination of the one or more fastening screws 115 and the one or more fastening tabs 113. In some cases, the one or more fastening tabs 113 are attached to or formed into the cranial hub 102. In some cases, the one or more fastening tabs 113 are configured to clip the cranial hub 102 to the frame 105. In some cases, the one or more fastening screws 115 are configured to create a tension between the one or more fastening tabs 113 and the frame 105. In some cases, the tension can be generated by using a slotted hole for the one or more fastening screws 115. In some cases, the frame 105 can include eight flanges 107 of different lengths with two flanges 107 on each edge or side of the central region of the frame 105. In some cases, each flange 107 can include a hole for inserting a fastener (e.g., a screw) to attach the cranial hub 102 to the skull of a subject via the holes of the flanges 107. In some cases, the frame 105 can include one or more windows 109 (e.g., 6 windows) configured to view some or all regions of the cranial hub 102 from above the frame 105. In some cases, the frame 105 can be made of a material such as Titanium. FIG. 22B depicts a superior view of the cranial hub 102 including the frame 105, the one or more flanges 107 (e.g., 6 flanges), one or more windows 109 (e.g., 6 windows), one or more fastening screws 115 (e.g., 3 screws), and one or more fastening tabs (e.g., 1 tab, not shown for clarity). In some cases, the frame 105 can be attached to the cranial hub 102 by a combination of the one or more fastening screws 115 and the one or more fastening tabs 113. In some cases, the one or more fastening tabs 113 are attached to or formed into the cranial hub 102. In some cases, the one or more fastening tabs 113 are configured to clip the cranial hub 102 to the frame 105. In some cases, the one or more screws are configured to create a tension between the one or more fastening tabs 113 and the frame 105. In some cases, the tension can be generated by using a slotted hole for the one or more screws. In some cases, the frame 105 can include eight flanges 107 of different lengths with two flanges 107 on each edge or side of the central region of the frame 105. In some cases, each flange 107 can include a hole for inserting a fastener (e.g., a screw) to attach the cranial hub 102 to the skull of a subject via the holes of the flanges 107. In some cases, the frame 105 can include one or more windows 109 (e.g., 6 windows) configured to view some or all regions of the cranial hub 102 from above the frame 105. In some cases, the frame 105 can be made of a material such as Titanium.

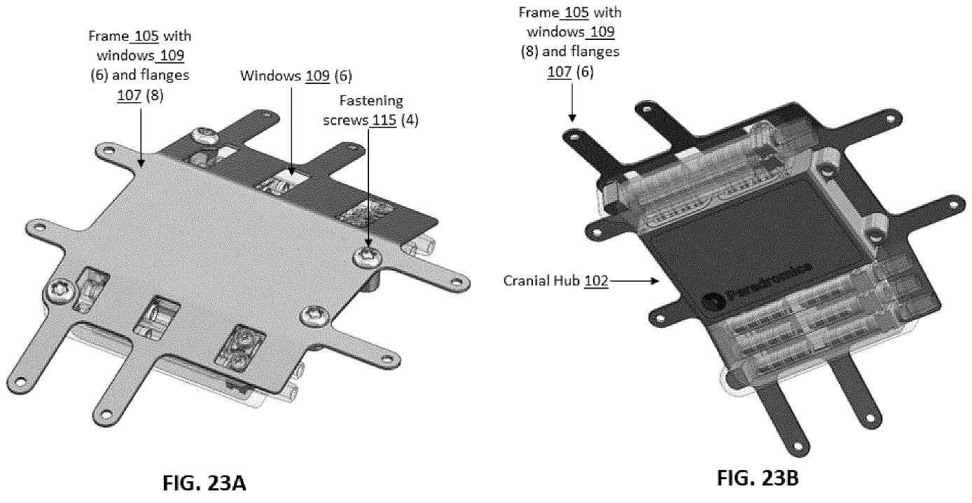

FIGS. 23A-23B depict a cranial hub 102 including a frame 105 with one or more viewing windows 109 and flanges 107 configured to attach the cranial hub 102 to a skull of a subject. In some cases, the frame 105 is configured to attach to the superior side of the cranial hub 102 with fasteners or tensioning screws. In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. FIG. 23A depicts a superior view of the cranial hub 102 including the frame 105, the one or more flanges 107 (e.g., 6 flanges), one or more windows 109 (e.g., 6 windows), and one or more fastening screws 115 (e.g., 4 screws). In some cases, the frame 105 can be attached to the cranial hub 102 by the one or more fastening screws 115. In some cases, the frame 105 can include eight flanges 107 of different lengths with two flanges 107 on each edge or side of the central region of the frame 105. In some cases, each flange 107 can include a hole for inserting a fastener (e.g., a screw) to attach the cranial hub 102 to the skull of a subject via the holes of the flanges 107. In some cases, the frame 105 can include one or more windows 109 (e.g., 6 windows) configured to view some or all regions of the cranial hub 102 from above the frame 105. In some cases, the frame 105 can be made of a material such as Titanium. FIG. 23B depicts the anterior view of the cranial hub 102 of FIG. 23A including the frame 105, the one or more flanges 107 (e.g., 6 flanges), one or more windows (e.g., 6 windows), and one or more screws (e.g., 4 screws).

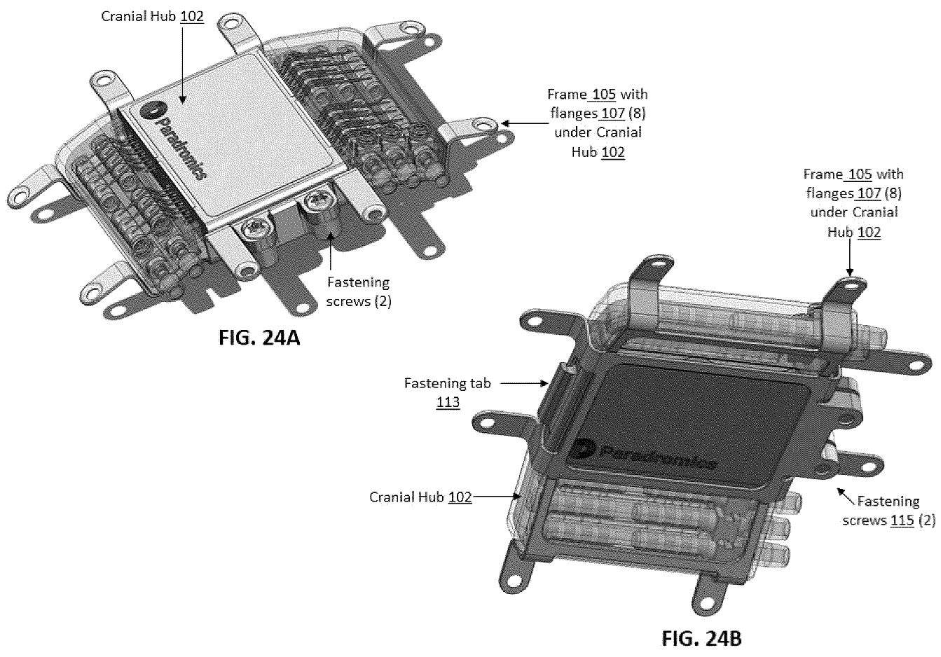

FIGS. 24A-24B depict a cranial hub 102 including a frame 105 that can include one or more flanges 107 configured to attach the cranial hub 102 to a skull of a subject. In some cases, the frame 105 is configured to attach to the anterior side of the cranial hub 102 using one or more fastening tabs 113 and one or more fastening screws 115. In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. Illustrated in FIG. 24A is a superior view of the cranial hub 102 with one or more flanges 107. In some cases, the frame 105 can be attached to the anterior side of the cranial hub 102. In some cases, the frame 105 can be attached to the anterior side of the cranial hub 102 and partially or completely wrap around to the superior side of the cranial hub 102. In some cases, the frame 105 can attach to the cranial hub 102 by a combination of one or more fastening tabs 113 (e.g., 1 tab) and one or more fastening screws 115 (e.g., 2 screws). In some cases, the one or more fastening tabs 113 are attached to or formed into the cranial hub 102. In some cases, the one or more fastening tabs 113 are configured to clip the cranial hub 102 to the frame 105. In some cases, the one or more fastening screws 115 are configured to further secure the frame 105 to the cranial hub 102. In some cases, the frame 105 can include eight flanges 107 of different lengths with two flanges 107 on each edge or side of the central region of the frame 105. In some cases, each flange 107 can include a hole for inserting a fastener (e.g., a screw) to attach the cranial hub 102 to the skull of a subject via the holes of the flanges 107. In some cases, the frame 105 can include one or more windows 109 (e.g., 6 windows) configured to view some or all regions of the cranial hub 102 from above the frame 105. FIG. 24B depicts the anterior view of the cranial hub 102 of FIG. 24A with one or more flanges 107. In some cases, the frame 105 can be attached to the anterior side of the cranial hub 102. In some cases, the frame 105 can be made of a material such as Titanium.

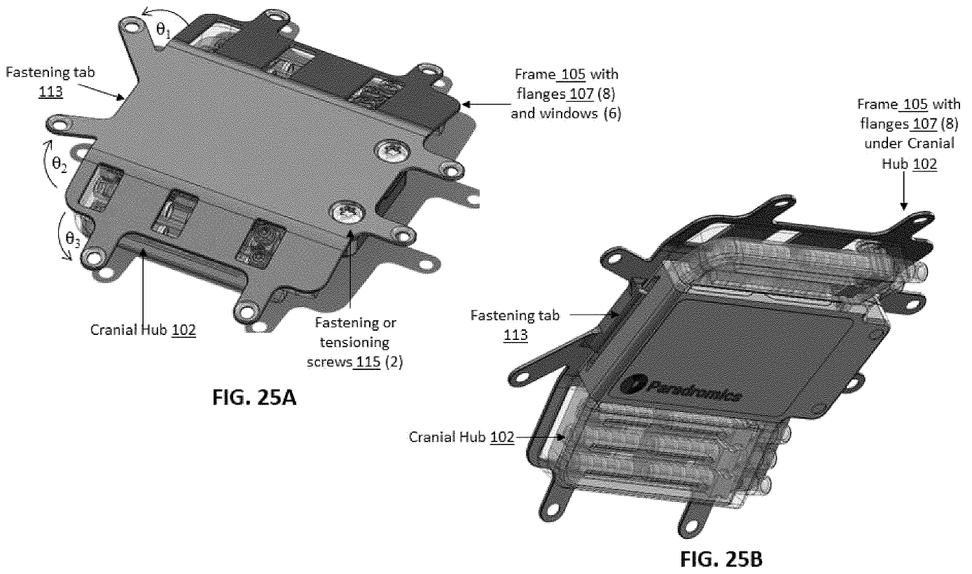

FIGS. 25A-25B depict a cranial hub 102 including a frame 105 with one or more viewing windows 109 and one or more flanges 107 configured to attach the cranial hub 102 to a skull of a subject. In some cases, the frame 105 is configured to attach to the superior side of the cranial hub 102 using one or more fastening tabs 113 and one or more fastening screws 115. In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. FIG. 25A depicts a superior view of the cranial hub 102 including the frame 105, the one or more flanges 107 (e.g., 6 flanges), the one or more windows 109 (e.g., 6 windows), the one or more fastening screws 115 (e.g., 2 screws), and one or more fastening tabs 113 (e.g., 1 tab). In some cases, the frame 105 can be attached to the cranial hub 102 by a combination of the one or more fastening screws 115 and the one or more fastening tabs 113. In some cases, the one or more fastening tabs 113 are formed into the cranial hub 102. In some cases, the one or more fastening tabs 113 are configured to clip the cranial hub 102 to the frame 105. In some cases, the one or more screws are configured to create a tension between the one or more fastening tabs 113 and the frame 105. In some cases, the tension can be generated by using a slotted hole for the one or more screws. In some cases, the frame 105 can include eight flanges 107 of different lengths with two flanges 107 on each edge or side of the central region of the frame 105. In some cases, any one of the flanges 107 can be formed at different angles (e.g., θ₁, θ₂, θ₃, and so on) relative to an edge or side of the frame 105. In some cases, each flange 107 can include a hole for inserting a fastener (e.g., a screw) to attach the cranial hub 102 to the skull of a subject via the holes of the flanges 107. In some cases, the frame 105 can include one or more windows 109 (e.g., 6 windows) configured to view some or all regions of the cranial hub 102 from above the frame 105. In some cases, the frame 105 can be made of a material such as Titanium. FIG. 25B depicts the anterior view of the cranial hub 102 of FIG. 25A including the frame 105, the one or more flanges 107 (e.g., 6 flanges), the one or more windows 109 (e.g., 6 windows), the one or more fastening screws 115 (e.g., 2 screws), and the one or more fastening tabs 113 (e.g., 1 tab). In some cases, the frame 105 can be made of a material such as Titanium.

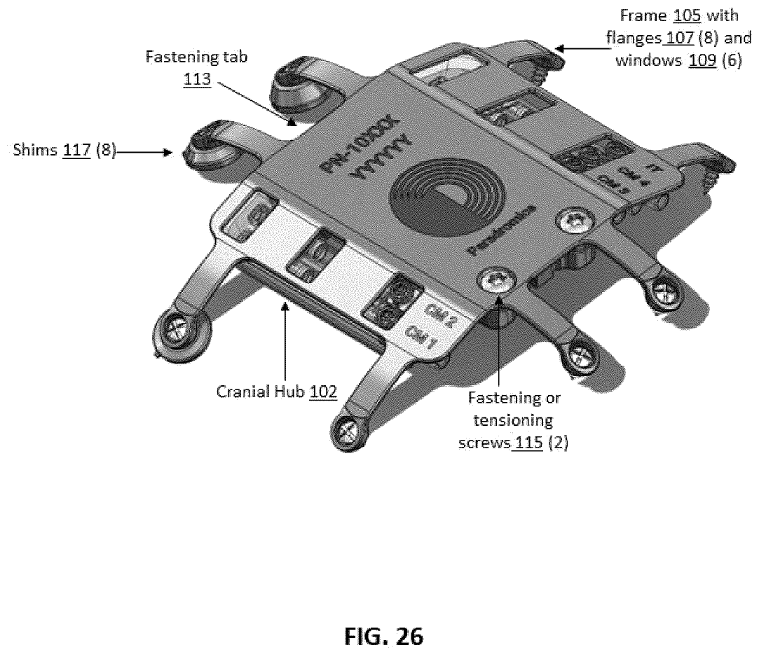

FIG. 26 depicts a cranial hub 102 including a frame 105 with one or more viewing windows 109 and one or more flanges 107 configured to attach the cranial hub 102 to a skull of a subject. In some cases, the frame 105 is configured to attach to the superior side of the cranial hub 102 for using one or more fastening tabs 113 and one or more fastening screws 115. In some cases, the frame 105 and the cranial hub 102 can be adjusted using one or more shims 117. In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. In some cases, the frame 105 can be attached to the cranial hub 102 by a combination of the one or more fastening screws 115 and the one or more fastening tabs 113 (not shown for clarity). In some cases, the one or more fastening tabs 113 are formed into the cranial hub 102. In some cases, the one or more fastening tabs 113 are configured to clip the cranial hub 102 to the frame 105. In some cases, the one or more fastening screws 115 are configured to create a tension between the one or more fastening tabs 113 and the frame 105. In some cases, the tension can be generated by using a slotted hole for the one or more fastening screws 115. In some cases, the frame 105 can include eight flanges 107 of different lengths with two flanges 107 on each edge or side of the central region of the frame 105. In some cases, any one of the flanges 107 can include a shim 117 configured to position the cranial hub 102 at an optimal location in the skull of the subject e.g., sit in a lower or higher profile relative to the skull. In some cases, each flange 107 can include a hole for inserting a fastener (e.g., a screw) to attach the cranial hub 102 to the skull of a subject via the holes of the flanges 107. In some cases, the frame 105 can include one or more windows 109 (e.g., 6 windows) configured to view or access some or all regions of the cranial hub 102 from above the frame 105. In some cases, frame 105 can be labeled with the position of the connector row 302, e.g., CM1, CM2, and CM3, and position of the link interface 305, e.g., IT. In some cases, the frame 105 can be made of a material such as Titanium.

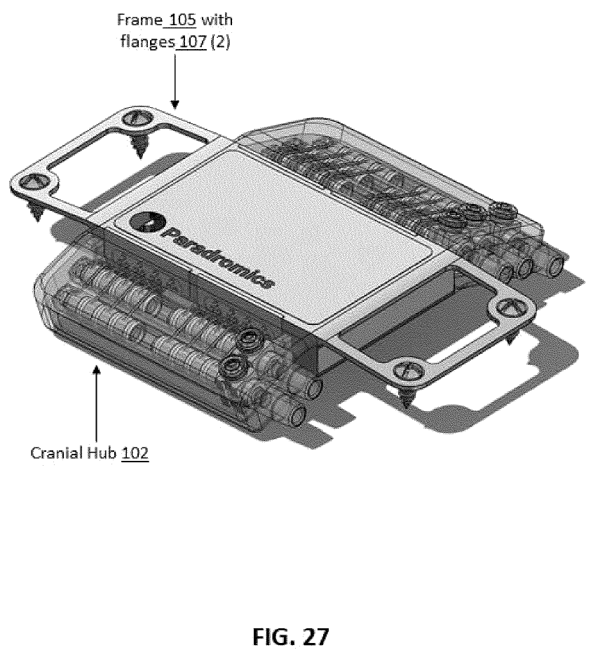

FIG. 27 depicts a cranial hub 102 including a frame 105 with one or more flanges 107 configured to attach the cranial hub 102 to a skull of a subject. In some cases, the frame 105 is configured to attach to the superior side of the cranial hub 102 using an adhesive, a snap fit, or welding. In some cases, the frame 105 or the flanges 107 can be formed into (e.g., molding) the cranial hub 102. In some cases, the frame 105 or the one or more flanges 107 can be formed or shaped to the skull of the subject before, during, or after surgery. In some cases, the frame 105 or the flanges 107 can be custom manufactured from preoperative CT or MRI data to match to patient's skull anatomy. In some cases, the frame 105 or the flanges 107 can be formed from a material such as a metal, a polymer, or an epoxy. In some cases, the frame 105 can include two flanges 107 of a same length with one flange 107 on an edge or side and another flange 107 on an opposite edge or side of the central region of the frame 105. In some cases, each flange 107 can include two hole for inserting a fastener (e.g., a screw) to attach the cranial hub 102 to the skull of a subject via the holes of the flanges 107. In some cases, the frame 105 can be made of a material such as Titanium.

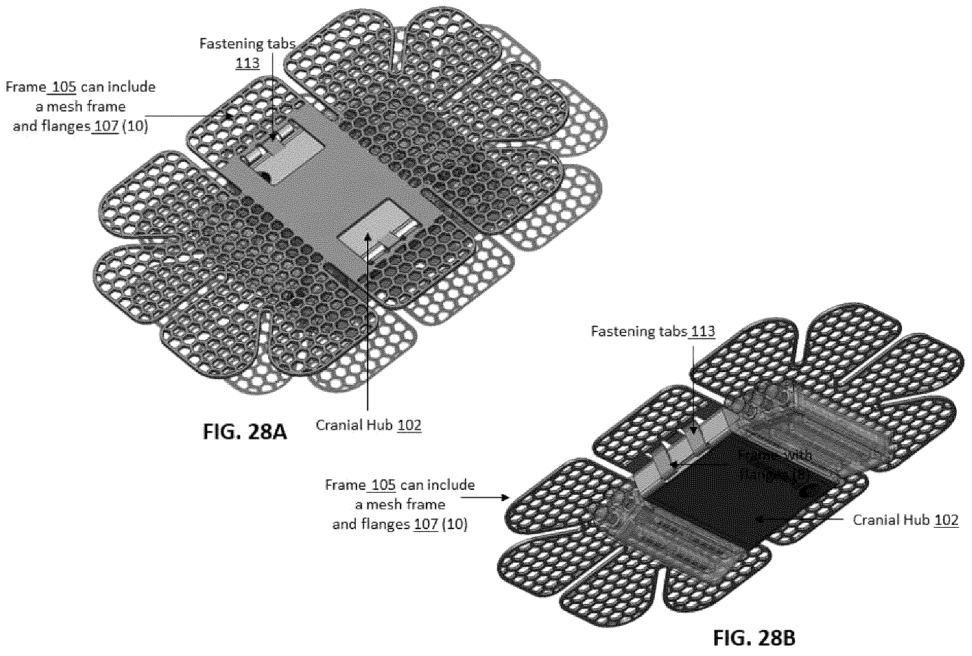

FIGS. 28A-28B depict a cranial hub 102 including a mesh frame 105 with one or more mesh flanges 107 configured to attach the cranial hub 102 to a skull of a subject. In some cases, the mesh frame 105 is configured to attach to the superior side of the cranial hub 102. In some cases, the attaching can include a snap fit using one or more fastening tabs 113. In some cases, the mesh frame 105 or the one or more mesh flanges 107 can be formed or shaped to the skull of the subject before, during, or after surgery. In some cases, the mesh frame 105 or the mesh flanges 107 can be custom manufactured from preoperative CT or MRI data to match to patient's skull anatomy. In some cases, the mesh frame 105 or the mesh flanges 107 can be formed from a material such as a metal, a polymer, or an epoxy. Illustrated in FIG. 28A is a superior view of the mesh frame 105 that can be attached to or formed into the superior side of the cranial hub 102. In some cases, the mesh frame 105 can be attached to the cranial hub 102 by one or more fastening tabs 113 (e.g., 4). In some cases, the mesh frame 105 can include ten mesh flanges 107 of a same or different shape or length. In some cases, three mesh flanges 107 can be located on each of two opposite edges or sides of the central region of the mesh frame 105. In some cases, two mesh flanges 107 can be located on each of two opposite edges or sides of the central region of the mesh frame 105. In some cases, each mesh flange 107 can include one or more holes (e.g., the mesh itself can be used for the holes) for inserting one or more fasteners (e.g., screws) to attach the cranial hub 102 to the skull of a subject via the holes of the mesh flanges 107. In some cases, the mesh frame 105 can be made of a material such as polyethylene. FIG. 28B depicts the anterior view of the cranial hub 102 of FIG. 28A including the mesh frame 105, the one or more mesh flanges 107, and the one or more fastening tabs 113.

### Cortical Module

Neural information may be collected using a cortical module (e.g., neural interface probe) implanted into the brain. In some embodiments, neural activity may be stimulated using a cortical module. In some embodiments, the cortical module may comprise a plurality of wires (e.g., electrodes), as described elsewhere herein. In some embodiments, the plurality of wires may comprise a flexible distal portion configured to interface with neural matter. In some embodiments, a wire may be referred to as an electrode.

The neural information may include measured waveforms such as extracellular neuronal recordings and/or intracellular neuronal recordings. In some embodiments, an electrode array in a cortical module may comprise a plurality of microwires that may be bonded to a readout integrated circuit (ROIC). In other embodiments, the electrodes may comprise patterned silicon probes or electrocorticography (ECOG) nets. In some instances, the ROIC and the electrodes may be fabricated from the same silicon substrate.

In some embodiments, the cortical module detects neural action potential events from many hundreds, thousands, tens of thousands, hundreds of thousands, or millions of neurons. In some embodiments, the events arise from extracellular measurements; in other embodiments, they arise from intracellular measurements. In some embodiments, the events arise from many hundreds, thousands, tens of thousands, hundreds of thousands, or millions of electrodes. In some embodiments, the cortical module may be embedded in neural tissue.

The cortical module may comprise complementary metal oxide semiconductor (CMOS) technology. In some embodiments, a circuit (or a discrete event detector) may be implemented in the CMOS electronic circuitry of an active pixel sensor in a ROIC of a cortical module. The discrete event detector may be configured to perform feature extraction or compressed sensing. Each active pixel may include one or more event detection circuits comprising an analog circuit and low-resolution analog-to-digital converter(s). Event detection circuits may be designed to detect specific temporal features in the voltage signal (“conditions” or events). Events (e.g., conditions) may include, for example: (1) the signal reaching a certain value (first threshold), (2) the derivative of the signal reaching a certain value (second threshold), or (3) a leaky integrator reaching a certain value (third threshold). A condition may also include a ratiometric criterion such as comparing the relative power in two frequency bands or some other normalized measurement. The condition(s) may be assessed at a sampling rate (for example, 1 kHz) considerably lower than the Nyquist sampling rate needed to fully capture the input signal.

The active pixel sensor may be defined by a width×height number of pixels, each of which contains a bank of condition circuits in the present disclosure. One example of a condition circuit may be a threshold filter.

The in-pixel detection circuitry may be configured to convert the measured analog voltage-over-time signal into a series of digital events (discrete events), each of which may be comprised of one or more bits summarizing salient aspects of the signal. The circuitry may be designed to detect features of the action potential waveform so that the event record captures all information about the neural activity.

A series of discrete events may be transmitted using relatively low voltage and power (compared to analog), thus reducing the total amount of power dissipated in the implant and decreasing the health risk to the recipient of the implanted cortical module.

Using the system in accordance to some embodiments, the action potentials may be resolved to within 1 ms timing resolution. Since each electrode may measure neural signals from a plurality of different neurons (such as 8 different neurons), a minimum sampling rate of 4 bits*1,000 samples per second=4 kb/s may distinguish between the different neuron sources. Allowing for some buffer (e.g., of up to 4 additional bits), this may translate to 4-8 Gb/s for 1 million electrodes, which may be a data rate that may be supported by commercial wireless technology, such as 60 GHz transceiver technology. In an example calculation of the data transmission requirements, one or more score values (such as 2-3 score values) per spike may be reported when sampling event values from transformed input signals. A spike may refer to a feature of a voltage-time waveform recorded from at least one neuron by the system in accordance with some embodiments. Further description of spikes can be found in U.S. Patent Application Publication No. 2019/0246929 A1 herein incorporated by reference in its entirety. For an average firing rate of 5 Hz, an average of 8 neurons per electrode, and a bit depth of 10 bits, 8*10 bits*5 samples per second=0.4 kb/s may distinguish between neuron sources. This may translate to 0.4 Gb/s for 1 million electrodes, which may be a data rate that is supported by commercial wireless technology, such as 60 GHz transceiver technology.

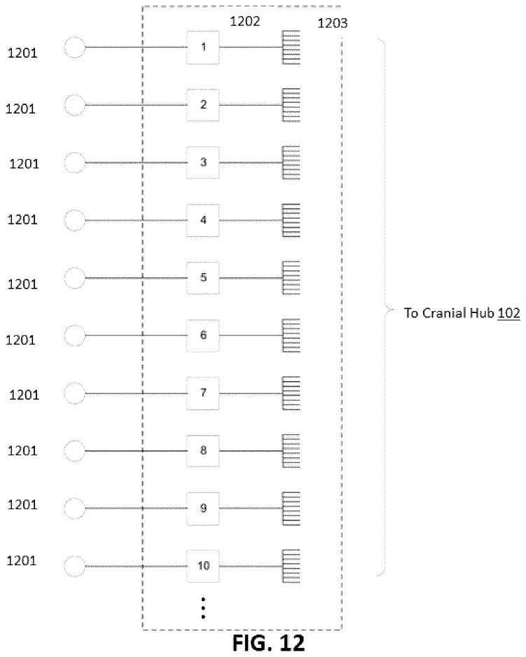

FIG. 12 illustrates a schematic of a highly parallel array of discrete event detectors configured to detect a plurality of discrete events from the firing of a plurality of neurons, in accordance with some embodiments. In some embodiments, a discrete event detector may refer to a circuit comprising an active sensor array. In some embodiments, the active sensor array may comprise a CMOS device. Referring to FIG. 12, a plurality of neural signals may be detected using a plurality of electrodes 1201 implanted in neural tissue. The plurality of electrodes may be part of a cortical module. The neural signals may be indicative of extracellular membrane potentials or intracellular membrane potentials. The plurality of electrodes may be electronically connected to a plurality of discrete event detectors 1202. In some embodiments, the plurality of discrete event detectors may be included as part of a cortical module. In some embodiments, the plurality of discrete event detectors may be included as part of a cranial hub. The plurality of discrete event detectors may be identical. In some embodiments, two or more of the discrete event detectors may be different. The discrete events recorded by the plurality of discrete event detectors may be transmitted via a plurality of signal lines (e.g., leads) 1203. The discrete events may be transmitted via the plurality of signal lines to a cranial hub (e.g., feature-event coalescence module). The plurality of discrete event detectors may be implemented in parallel using CMOS technology, or any other electronic circuit topologies or semiconductor fabrication technologies.

In some embodiments, a cortical module may be referred to as a neural probe. In some embodiments, the cortical module may comprise a patterned silicon probe. In some embodiments, the cortical module may comprise a microwire bundle.

In some embodiments, adjacent electrodes of a cortical module may record the same bioelectric event (e.g., action potential from a single neuron), but the event may produce a different signature voltage waveform on each electrode/channel. In some embodiments, combining logic may coalesce the simple events detected across several channels into one event, reflecting this characteristic.

In some embodiments, the digital signal from the ROIC or other instantiation of the cortical module 101, cranial hub 102 or wireless transmitter 103 may be transmitted out of the body using a low-energy wavelength of electromagnetic radiation that may be compatible with transmission through a few millimeters or a few centimeters (e.g., 1-20 mm) of biological tissue. Examples may include 60 GHz transceivers or VCSEL-based infrared transceivers. In such an embodiment, computation may be minimized within the body, since the feature-event coalescence module and the approximator module are located outside of the body. Accordingly, heat dissipation caused by computation may be reduced within the body. In the above example, the feature-event coalescence module may be implemented via GPGPU or CPU processing.

In some embodiments, the cortical module may receive neural signals from a plurality of wires (e.g., electrodes or microelectrodes) which have been implanted into deep neural matter or superficial neural matter. In some embodiments, the cortical module 101 may comprise at least one electrode 104. In some embodiments, the cortical module may comprise a parallel electrode array. In some embodiments, the parallel electrode array may comprise a plurality of microwires bonded to a readout integrated circuit (ROIC), and wherein the ROIC may comprise a plurality of CMOS pixel sensors. In some embodiments, distal ends of microwires (e.g., wires or electrodes) may be flexible and in contact with neural matter when the cortical module is implanted into a brain. In some embodiments, the neural signal waveforms may be extracellular or intracellular action potential waveforms obtained by the implanted cortical module. The cortical module (e.g., a neural interface probe comprising a parallel electrode array) is further described in U.S. Patent Application Publication Nos. 2019/0246929 and 2020/0046240, where both patent application publications are herein incorporated by reference in their entirety.

As illustrated in FIG. 1, a DDI system may include one or more cortical modules 101 configured to detect a plurality of signals in a human brain. In some embodiments, the DDI system may include a plurality of cortical modules 101. The plurality of cortical modules may include at least about one, two, three, four, or more cortical modules. The plurality of signals may be associated with electrical or chemical signals from a plurality of neurons in the human brain. A cortical module may include a plurality of sensors having at least about one, one-hundred, two-hundred, three-hundred, four-hundred, or more sensors configured to detect the plurality of signals. A cortical module may further be configured to perform a plurality of operations on the plurality of signals. The plurality of operations may include a plurality of signal processing operations, for example, signal amplification, analog to digital conversion (ADC), or signal modulation.

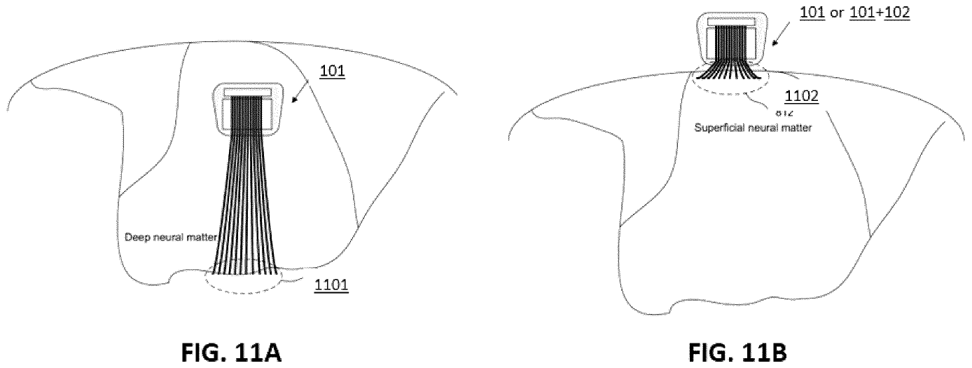

FIGS. 11A-11B illustrates examples of cortical modules 101 implanted in different regions in a brain, in accordance with some embodiments. FIG. 11A depicts a schematic of a cortical module implanted deep within a brain (e.g., deep neural matter) 1101, in accordance with some embodiments. In some embodiments, the cortical module may be inserted into the deep-tissue regions of the brain of a test subject. During insertion of the probe, the free ends of the wires spread out within the brain tissue such that the electrodes deploy in a three-dimensional arrangement over a deep-brain target area 1101. FIG. 11B depicts a schematic of a cortical module (e.g., neural-interface probe) implanted on a superficial target on a brain (e.g., superficial neural matter) 1102, in accordance with some embodiments. The probe may be inserted onto a superficial tissue region of the brain of a test subject. The tissue region may, for example be a cortical region of the brain. In some embodiments, where the cortical module may be implanted on the tissue region, the free ends of the wires spread out such that the electrodes deploy in a three-dimensional arrangement over the tissue region. The system may also be configured to be implanted in regions of the peripheral nervous system, such as the vagus nerve.

In some examples, the cortical module (e.g., neural-interface probe) 101 has a high aspect ratio since it may be used in deep brain regions as depicted in in FIG. 11A. In some embodiments, as depicted in FIG. 11B, the cortical module in configured for use in shallow or superficial brain regions and may comprise a low aspect ratio. In some embodiments, a length of a cortical module (including the electrode length, or flexible distal portion) may range from 1 cm to about 8 cm. In some embodiments, the length of a cortical module including the electrode (e.g., wire or flexible distal portion) may be 1 cm or less. In some embodiments, the length of the cortical module including the electrode (e.g., wire or flexible distal portion) may be 8 cm or more. Accordingly, cortical modules of different lengths and other dimensions (width, thickness, etc.) may be used for different regions of the brain in accordance with various embodiments. In some embodiments, a cortical module may be used to implement a method for monitoring and/or stimulating neural activity. In some embodiments, the method may comprise inserting the cortical module into a brain, such that the flexible distal portion of the wires interfaces and may be in contact with an area of the neural matter. The method may further comprise monitoring and/or stimulating neural activity in the area via a plurality of electrical signals transmitted between the cortical module and the neural matter. The plurality of electrical signals may be transmitted through the plurality of wires. In some embodiments, the method may further comprise transmitting the electrical signals from the cortical module to the cranial hub described herein, via one or more wireless or wired communication channels.

Described herein are various embodiments of cranial hub interfaces. In some embodiments, the cranial hub interfaces comprise physical interfaces. In some embodiments physical interfaces may be implemented using Bal Seal® connectors to allow connections to custom leads.

A cortical module 101 may be configured to transmit a signal to a cranial hub 102. In some embodiments, a cortical module may comprise a plurality of cortical modules. In some embodiments, a cortical module may comprise a plurality of cortical modules, where each cortical module of the plurality may have a different location. A cortical module may be configured to transmit a signal to a cranial hub, where the signal may comprise a plurality of signals. In some embodiments, a cortical module may transmit a signal to each cranial hub of a plurality of cranial hubs.

In some embodiments, a cortical module may comprise a passive cortical module. In some embodiments, a passive cortical module may refer to a cortical module where each wire (e.g., electrode) is hard wired. In some embodiments, hard wired may refer to an electrode that is directly wired to the hub (e.g., Utah Array, see later in the paragraph). In some embodiments, hard wired may refer to a lead directly connected to the hub, for example by soldering without Bal Seal® connectors. In some embodiments, a passive cortical module refers to a cortical module that may not have onboard electronics. In certain examples, the lead from the cranial hub is directly linked to the wire (e.g., electrode) and there is no sensor (e.g., discrete event detector, sensor array, CMOS, etc . . . ) in the cortical module. In some embodiments, the passive cortical module is an off-the-shelf product. In some embodiments, the passive cortical module is a Utah Array™. In some embodiments, the passive cortical module is a Utah Array™ having 96 individual wires (e.g., electrodes).

### Wireless Transmitter

In some embodiments, the internal wireless implant may comprise a field programmable gate array (FPGA), a battery, an AC-DC conversion and power electronics unit and a light source. In some embodiments, the light source may be used to transmit data wirelessly. In some embodiments, the light source may comprise a laser and a laser driver. In some embodiments, the laser may be a vertical-cavity surface-emitting laser (e.g., VCSEL). In some embodiments, the VCSEL may comprise a wavelength of about 850 nm. In some embodiments, the laser driver may be a VCSEL driver.

The plurality of wireless transmitters 103 may include at least about one, two, three, four, or more wireless transmitters (e.g., implantable wireless transmitters). Additional details of the wireless transmitter are described herein.

The wireless transmitter 103 may be configured to wirelessly transmit the plurality of signals to an external device for further processing of the plurality of signals. Wireless transmission of the plurality of signals may be performed by an optical transmitter of the wireless transmitter. The plurality of signals may then be considered optical signals. The optical transmitter may include, for example, a semiconductor laser diode. The semiconductor laser diode may include, for example, a VCSEL. An external device may be configured to wirelessly receive the plurality of optical signals from the optical transmitter of the wireless transmitter. Wireless receiving of the plurality of optical signals may be performed by, for example, an optical receiver of the external device. The optical receiver may include, for example, a photodiode. The optical receiver may include, for example, a silicon photomultiplier array. The plurality of optical signals received by the optical receiver may be associated with a plurality of telemetry signals for aligning the inductive link described elsewhere herein. The plurality of telemetry signals may include, for example, telemetry signals for positional alignment of the inductive link. The plurality of telemetry signals for positional alignment may be, for example, signals for fine positional alignment of the inductive link.

The wireless transmitter 103 may be configured to wirelessly receive a plurality of signals from an external device. The plurality of signals may be received through, for example, an inductive link between the wireless transmitter and the external device. In some embodiments, the plurality of signals may be transmitted through, for example, an inductive link between the wireless transmitter and the external device. In this sense, the inductive link may also serve as a communications link. The inductive link may include, for example, an inductive link configured to transmit a plurality of signals to the external device. The inductive link may include, for example, an inductive link configured to receive a plurality of signals from the external device. The plurality of signals may include instructions for programming the plurality of wireless transmitters, the plurality of cranial hubs 102, or the plurality of cortical modules 101. The plurality of signals may include, for example, signals for aligning the inductive link between the wireless transmitter and the external device. The plurality of signals may be associated with telemetry signals for aligning the inductive link described elsewhere herein. The telemetry signals may be associated with, for example, positional alignment of the inductive link. The telemetry signals may be associated with, for example, temperatures associated with the inductive link. The telemetry signals for positional alignment may be, for example, signals for coarse positional alignment of the inductive link.

The wireless transmitter 103 may be configured to wirelessly receive power from an external device. The received power may be received through, for example, an inductive link between the wireless transmitter and the external device. The inductive link may include, for example, an inductive link configured to receive power from the external device. In this sense, the inductive link may be an inductive power link. The received power may be used to, for example, power the wireless transmitter. The received power may be used to, for example, charge a power source associated with the wireless transmitter. The received power may further be used to, for example, provide power for a plurality of other wireless transmitters. Other wireless transmitters may include, for example, a plurality of cranial hubs 102 and a plurality of cortical modules 101. The inductive power link may be aligned between the wireless transmitter and the external device. The inductive power link may be aligned using, for example, a plurality of signals. The plurality of signals may include, for example, signals for aligning the inductive power link between the wireless transmitter and the external device. The plurality of signals may be associated with telemetry signals for aligning the inductive power link. The telemetry signals may be associated with, for example, positional alignment of the inductive power link. The telemetry signals may be associated with, for example, temperatures associated with the inductive power link. The telemetry signals for positional alignment may be, for example, signals for coarse positional alignment of the inductive power link.

The external device that may be configured to wirelessly transmit or receive signals to or from the wireless transmitter 103 may be further configured to also wirelessly transmit power to the wireless transmitter. In this sense, the external device may be an integrated external device for both wireless communication and wireless power transmission.

In some embodiments, the cranial hub 102 may comprise at least one wireless transmitter.

### Methods

### Methods of Operation

FIG. 13 depicts a cranial hub diagram comprising a wireless transmitter (e.g., transceiver) interface in accordance with some embodiments. In some embodiments, the cranial hub may comprise methods (e.g., functions or protocols) for power distribution, slow control, synchronization/clock distribution, data collection, processing, readout, or a combination thereof.

In some embodiments, a power distribution method may comprise receiving power from the wireless transmitter (e.g., transceiver implant) 103 and delivering power to at least one cortical module 102. In some embodiments, a power distribution method may comprise receiving power form the wireless transmitter and delivering power to one, two, three, four or more cortical modules.

In some embodiments, a cranial hub power distribution method may comprise receiving power form the wireless transmitter (e.g., transceiver implant) and delivering power to at least one of eight cortical modules. In some examples, the power distribution method may comprise delivering power to each of a plurality of cortical modules independently. In some embodiments, the power distribution method may comprise monitoring current to allow automatic shutoff of cortical modules.

In some embodiments, a slow control method may comprise routing, commanding, and controlling data transmitted to or from the wireless transmitter (e.g., transceiver implant) either locally (e.g., within the cranial hub) or to the plurality of cortical modules.

In some embodiments, methods may comprise synchronization. In some embodiments, synchronization methods may comprise clock distribution. In some embodiments, clock distribution methods may comprise broadcasting commands to at least one cortical module simultaneously for synchronous operations (e.g., starting of recording). In some embodiments, synchronization and/or clock distribution methods may comprise allowing the cranial hub to serve as a master clock. In some embodiments, the master clock (e.g., cranial hub) may allow for synchronous operation of each of the plurality of cortical modules. In some embodiments, timing markers can be distributed from the cranial hub to cortical modules that are running asynchronously.

In some embodiments, methods may comprise collecting data, processing data, reading out data, or a combination thereof. In some embodiments, the cranial hub data collecting methods may comprise collecting data at up to 30Mbps from each of the plurality of cortical modules. In some embodiments, data processing methods comprise reducing data on the cranial hub comprising a field programmable gate array (FPGA) such that only active channels are sent for transmission.

### Methods of Implantation

In some embodiments, a method for implantation may comprise implanting the cranial hub 102 into a craniotomy (e.g., surgically removed hole of a human skull) and connecting the cranial hub to the at least one cortical module 101 as depicted in FIG. 1. In some embodiments, methods of implanting a cranial hub may comprise implanting the cranial hub into the craniotomy of the skull of a subject to connect the cranial hub to the implanted wireless transmitter 103. In some embodiments, the implantation method may comprise connecting a lead 501 from a cortical module to a contact within the cranial hub. In some embodiments, the method comprises: obtaining or providing a cranial hub and at least one cortical module; implanting the cranial hub within a craniotomy of a skull; and securing a lead 501 from the cortical module to a connector of the cranial hub. In some embodiments, a method of implantation may comprise connecting leads 501 to offset link interfaces 305 within the cranial hub; inserting the most central lead 501 first; and tightening the set screw.

In some embodiments, an implantation method may comprise detaching a lead 501 to the cranial hub 102 from a cortical module to allow for replacement of the cranial hub, where the cranial hub may be implanted in the skull of a patient. In some embodiments, the leads 501 may be detached from the cranial hub to allow for replacement of at least one cortical module 101, where the cranial hub remains implanted in the skull.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” refers to A alone, B alone, C alone, A and B together, A and C together, B and C together, or A, B and C together.

### EXAMPLES

The following illustrative examples are representative of embodiments of devices, systems, and methods described herein and are not meant to be limiting in any way.

### Example 1: Hub with an 18-Degree Bend Angle

This example describes a cranial hub 102 designed to fit within a craniotomy (e.g., surgically removed hole within a human skull). The cranial hub in this example, as shown in FIG. 3A, includes a sealed central body 304, five connector rows 302 and a header 301. Four of the five connector rows are configured to connect cortical modules 101 to the PCB 401 (e.g., processor) of the cranial hub. The fifth connector row is configured to connect the cranial hub to the internal wireless transmitter 103 implant. Each of all five connector rows comprise 8 individual contacts 301. Each contact 303 allows for transmission of power or a communication signal. FIG. 3B depicts a side profile of the cranial hub, including the link interface 305 of each of the five connector rows. FIG. 3B also depicts the side profile of the sealed central body and the header. FIG. 4 depicts an exploded view of the hub comprising the PCB 401 and hermetic feedthroughs 402 for connecting the contacts 303 of the connector rows 302 to the PCB 401 inside the sealed central body 304. The PCB 401 is configured for power management, clocking, multiplexing signals from cortical modules 101.

FIG. 7A depicts a top-down view, scaled drawing of the cranial hub. In this example, the overall housing length 705 of the cranial hub is 48.7 mm; the housing width 704 is 33.5 mm, the total overall width including connector row length 703 is 37.8 mm, the center-to-center distance between the link interface 701 is 3.8 mm and the center-to-center spacing of the link interfaces on either side of the sealed central body 702 is 30.2 mm. In this example, the contact pitch 708 (e.g., center-to-center contact spacing) is 2.3 mm as shown in FIG. 7B. FIG. 7C depicts a scaled drawing of the side profile of the cranial hub. As shown in FIG. 7C, the housing thickness 707 as measured at the center point of the sealed central body 304 is 6.1 mm and the housing thickness 707 as measured at the edge of the header 301 is 6.0 mm. The angle (e.g., bend angle 706) between a horizontal line running through the plane of the top surface of the sealed central body 304 relative to the top surface of the header 301 is 18°. The position of the bend angle is 13.6 mm from the left edge of the header as depicted in FIG. 7C. The position of the bend angle from the far-right edge of the header width is 9.8 mm as depicted in FIG. 7C.

### Example 2: Wireless Cranial Hub

The purpose of this example is to describe a cranial hub 102 incorporating wireless power and data transmission through the skin 103 of a subject. In this example, linking leads 501 from a separate wireless transmitter 103 to the cranial hub, during implantation surgery, is not required. One or more cortical modules connect to the cranial hub, where the cranial hub communicates wirelessly with an external device (e.g., device external to human body). The cranial hub includes a battery to support operation during power disruptions. Additionally, the cranial hub may be located over the location of the cortical modules or may be located in an ergonomic location such as behind the ear. In this example, a wearable component provides through skin power and data connectivity.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. An implantable cranial hub device comprising:
a. a housing comprising a curve, the curve comprising a radius of curvature;
b. a plurality of connector rows, wherein the plurality of connector rows is configured to operably couple to a plurality of implantable cortical modules and at least one wireless transmitter module;
c. electronic circuitry configured to (1) aggregate a plurality of signals received from the plurality of implantable cortical modules and (2) transmit the aggregated plurality of signals to the at least one wireless transmitter; and
d. a frame configured to permit the cranial hub to be mounted to a skull of the subject.

2. The device of claim 1, wherein the radius of curvature is about 80 mm to about 120 mm.

3. The device of claim 1, wherein the curve comprises at least one portion that is bent and at least one portion that is flat.

4. The device of claim 3, wherein the at least one portion that is bent comprises a bend angle, θ.

5. The device of claim 4, wherein the bend angle, θ, is about 15° to 20°.

6. The device of claim 1, wherein the frame is configured to be formed to the skull of the subject before or during a surgery of the subject.

7. The device of claim 6, wherein the frame is configured, using data from preoperative CT or MRI, to form to the skull of the subject.

8. The device of claim 1, wherein a size of the frame is configured to be reduced by trimming the frame.

9. The device of claim 1, wherein the frame attaches to the cranial hub on a superior side of the cranial hub, an anterior side of the cranial hub, a side of the cranial hub, or any combination thereof.

10. The device of claim 9, wherein the frame that attaches to the anterior side is configured to cradle the cranial hub and to wrap around the cranial hub to the superior side of the cranial hub.

11. The device of claim 1, wherein the frame attaches to the cranial hub using any one or more of welding, fasteners, or slip fits.

12. The device of claim 1, wherein a material of the frame comprises a formable material.

13. The device of claim 1, further comprising one or more flanges configured to be attached to or formed into the frame, the cranial hub, or a combination thereof.

14. The device of claim 13, wherein the one or more flanges are configured to permit the cranial hub to be mounted to the skull of the subject.

15. The device of claim 13, wherein the one or more flanges are configured to be formed to the skull of the subject before or during the surgery of the subject.

16. The device of claim 15, wherein the one or more flanges are configured using data from the preoperative CT or MRI to form to the skull of the subject.

17. The device of claim 13, wherein a material of the one or more flanges comprises a formable material.

18. The device of claim 1, further comprising one or more shims configured to position the frame at an optimal position relative the skull of the subject.

19. An implantable system comprising the implantable cranial hub device and the at least one wireless transmitter of claim 1.

20. The device of claim 1, further comprising a sensor, wherein the sensor is configured to receive signals from a passive cortical module.
